(19) United States
(12) Patent Application Publication
(10) Pub. No.: US 2025/0114219 A1
(43) Pub. Date: Apr. 10, 2025

(54) Unibody Endoskeletal Transtibial Prosthetic Devices and Digital Fabrication Workflow

(71) Applicants: Joshua PELZ, San Diego, CA (US); Luca DE VIVO, Oahu, HI (US); Herbert J. BARRACK, San Diego, CA (US); Falko KUESTER, La Jolla, CA (US)

(72) Inventors: Joshua PELZ, San Diego, CA (US); Luca DE VIVO, Oahu, HI (US); Herbert J. BARRACK, San Diego, CA (US); Falko KUESTER, La Jolla, CA (US)

(21) Appl. No.: 18/984,659

(22) Filed: Dec. 17, 2024

Related U.S. Application Data
(63) Continuation-in-part of application No. 18/581,180, filed on Feb. 19, 2024, now Pat. No. 12,208,025.
Application No. 18/581,180 is a continuation of application No. 18/009,915, filed on Dec. 12, 2022, now Pat. No. 11,938,043.
(60) Provisional application No. 63/137,268, filed on Jan. 14, 2021.

(51) Int. Cl. A61F 2/80; A61F 2/50; A61F 2/60; A61F 2/66; B33Y 50/00; B33Y 80/00

(52) U.S. Cl. CPC A61F 2/60; A61F 2/5046; A61F 2/6607; A61F 2/80; B33Y 50/00; B33Y 80/00; A61F 2002/505; A61F 2002/607; A61F 2002/6621; A61F 2002/6642; A61F 2002/6685

(57) ABSTRACT

Methods for producing a transtibial prosthetic device include generating a scan of a reference prosthetic limb, wherein the reference prosthetic limb has a reference socket, a reference pylon, and a reference foot. A digital model for the transtibial prosthetic device is generated, wherein the transtibial prosthetic device comprises i) a socket, ii) a pylon comprising a unitary truss structure, and iii) a foot-ankle complex. An alignment of the pylon with the socket and with the foot-ankle complex in the digital model is based on the scan. The method includes fabricating, using the digital model and 3D printing, the pylon and the foot-ankle complex as a unitary single piece.

### RELATED APPLICATIONS

This application is a continuation-in-part of U.S. patent application Ser. No. 18/581,180, filed on Feb. 19, 2024, and entitled “Unibody Endoskeletal Transtibial Prosthetic Devices and Digital Fabrication Workflow”; which is a continuation of and claims priority under 35 U.S.C. § 120 from U.S. patent application Ser. No. 18/009,915, filed on Dec. 12, 2022, issued as U.S. Pat. No. 11,938,043, and entitled “Unibody Endoskeletal Transtibial Prosthetic Devices and Digital Fabrication Workflow”; which is a U.S. National Phase under 35 U.S.C. § 371 of International Application No. PCT/US2022/011939, filed on Jan. 11, 2022; which claims priority under 35 U.S.C. § 119 and all applicable statutes and treaties from U.S. Provisional Application No. 63/137,268, filed on Jan. 14, 2021; all of which are incorporated by reference herein in their entirety.

### STATEMENT OF GOVERNMENT INTEREST

This invention was made with government support under grant number CNS-1338192 awarded by the National Science Foundation, under grant number 70NANB 17H211 awarded by the National Institute of Standards and Technology, and under grant number W912HZ172-0024 awarded by the US Army Research Office. The government has certain rights in the invention.

### BACKGROUND

In 2017, the World Health Organization (WHO) estimated that globally there were 35-40 million people in need of prosthetics or other assistive devices, with this number expected to double by 2050. Only 5-15% of people in need have access to prosthetics or other assistive devices, in both underserved and developed countries. As a result, a staggering 34-38 million people, 2.1 million of whom are in the United States, live restricted and often painful lives. Among the contributing medical conditions that are causing more than 185,000 Americans to lose limbs every year are vascular disease (54%), trauma (44%), and cancer (2%). People with diabetes are especially prone to vascular problems necessitating lower limb amputation, and this situation has been exacerbated by the COVID-19 pandemic. The elderly, particularly in aging populations with an increased incidence of diabetes, are especially at risk. Mobility and health-related issues resulting from amputation adversely affect patients' quality of life and, particularly in children and adolescents, may result in psychological and psychosocial issues as well. Recovery and rehabilitation are adversely affected by the cost and manufacturing time of prosthetics. This situation is particularly serious in developing countries where amputees often have limited access to medical facilities and experienced practitioners.

Conventional transtibial prosthetic devices are formed from multiple expensive components. Patient specific designs are typically achieved by manual molding, sculpting and composite lay-up to fabricate the socket, and then assembling an appropriate prosthetic from expensive 3^{rd}-party components.

The three basic components of a conventional transtibial prosthetic limb include the socket, pylon, and ankle-foot complex. The socket attaches the prosthesis to the wearer, such as via corsets, straps, lock-pins, or vacuums. Sockets are typically made from composite materials such as Carbon Fiber Reinforced Polymer (CFRP). The pylon is commonly made of CFRP or aluminum and is used to adjust height and transfer load between the socket and foot. Connections between socket, pylon, and ankle-foot complex can be made with pyramid connectors, which allow for proper trochanter (hip)-knee-ankle (TKA) line adjustment. Adjustable prosthetic devices are called endoskeletal systems and are very popular for a number of reasons, including greater flexibility, lower weight, improved gait performance and better comfort, among other things. If components are rigidly fixed together and cannot be aligned post-fabrication, the device is an exoskeletal system. The ankle-foot complex serves to interface with the ground and can be active or passive to provide proper gait mobility. Simpler passive systems are stiff and do not enable energy capture and release, which results in high metabolic cost for walking and an asymmetrical gait. High-performance passive systems use special materials and designs to store and release energy during the gait cycle to reduce metabolic cost of walking and increase comfort and functionality. Active systems use powered motors, servos, and sensors to control the gait cycle and can significantly enhance performance. However, active systems are prohibitively expensive, less reliable, require a power-source, and are very difficult to access for the majority of the world's amputee population.

The fitting process for a conventional prosthetic device is complex and expensive, requiring multiple steps. Prosthetists require many years, if not decades, of experience to be able to provide effective prosthetic care using traditional molding and sculpting processes. One of the main reasons amputees choose not to wear their device is an uncomfortable socket, and the socket will only be as good as the prosthetist who shapes it. Further, to solve the prosthetic accessibility crisis, it is estimated upwards of 75,000 more prosthetists are needed worldwide; training new prosthetists is an expensive time-consuming endeavor.

Designs of the ankle-foot complex have benefited from advancements in materials science and manufacturing technology. In general, whether the foot-ankle complex is active or passive, the top of the complex includes a horizontal edge to offer a connection with the pylon. The Solid Ankle Cushioned Heel (SACH) model is a simple passive prosthetic foot having a resilient foot inset. The static design of the SACH foot, results in asymmetric gait characteristics, high metabolic costs, and low energy return benefit.

While basic prosthetic foot models similar to the SACH model are commonly used, new materials and designs are emerging. New models include running-blades, the NIAGARA FOOT™ and the SEATTLE FOOT®, among others. Running-blades are carbon fiber blade-type feet that are designed for running, but suffer in walking situations. The, Niagara foot a futuristic-looking solid ankle foot, is made from HYTREL®. Hytrel is a thermoplastic polyester elastomer that provides superior durability and energy return. The Niagara Foot has been tested to more than 3,000,000 loading-cycles and shows increased durability over carbon fiber feet. Seattle Foot incorporates molded and manufactured components of various materials including carbon fiber, nylon, and metal. Seattle Foot can either be bolted directly to the pylon or attached with special adapters that simulate ankle articulation.

More recent efforts focus on digital fabrication and additive manufacturing. Summit U.S. Pat. No. 8,366,789 describes a method to create an exoskeletal prosthetic limb that can be simultaneously printed in one piece from a single material. Although the designs disclosed in this patent are novel, exoskeletal systems have lost favor over endoskeletal systems due to many limiting factors. One such important factor is the inability to adjust the design post-fabrication, which is a common problem for all amputees and even more important for new amputees whose amputated limb will change shape significantly during the first several months to 1 year. If exoskeletal devices are used for these newer amputees, the patient will either endure a painful, poorly functioning socket or must go back to the prosthetist to be remeasured and a new device constructed. One disclosed design includes elongated members that span the length of the lower leg having front and rear members that resemble the outer shape of the contralateral limb. Weight is distributed between the front and rear members. These members are coupled with a basic solid foot and are printed in a single photopolymer material. The use of a single material has significant disadvantages in functionality, because regions that would benefit from being stiff have to compromise with regions that should be flexible and compliant. Further, while the vat photopolymerization method, specified by Summit as the preferred printer type, can produce detailed parts with fine features, photopolymers generally have lower strength, flexibility, and durability compared to thermopolymers. Other disclosed designs include ball and socket connections between an elongated front member to the foot. Other designs include multiple mechanical linkages in the knee area and ball and sockets in the ankle area. The mechanical linkage and ball and socket connections are likely to wear and become loose after many cycles of movement, which will negatively affect the gait of a user. The simple foot and solid elongate supports that extend from knee to foot will be relatively thick and heavy to meet requirement for applied forces. The use of photopolymer material will lead to catastrophic failure if loads exceed material capability, and could result in a severely injured patient.

Layman et al. U.S. Pat. No. 10,010,433 and Layman et al. U.S. Pat. No. 9,480,581 describes imaging and digital processes to make a 3D (three-dimensional) printed socket. A check socket is first fabricated and matched with traditional alignment connectors, pylon, and foot/ankle componentry. Alignment and socket fit adjustment is achieved through manual methods requiring an experienced prosthetist and patient visits. A digital copy of the aligned device is then produced using a scanner. The digital copy is used to design a patient-fit socket with attachment points aligned for the specific patient. The printed socket may improve fit, but the time-consuming and manual fitting and alignment process will reduce the efficacy of this method. Further, the use of traditional hardware, connectors, pylon, and ankle-foot complex will increase cost and weight, while reducing time savings and customization afforded by a digital process.

Valenti, “Experience with Endoflex: A Monolithic Thermoplastic Prosthesis for Below-Knee Amputees,” Journal of Prosthetics and Orthotics, Volume 3, Number 1, pp. 43-50 describes monolithic socket and rod-shaped solid pylon that can be fitted to a prosthetic foot.

The creation and fitting of prosthetic devices remain expensive. Most patients are fitted with prosthetic devices that are formed from separate and expensive components. Separate components add weight, complexity, and expense.

Recently, a Japanese company Instalimb has used 3D-CAD, 3D printing and machine learning (AI) technology to create a 3D printed transtibial prosthetic. The printed prosthesis is a printed socket parts that is then joined with a standard solid pylon and foot. The printed socket offers a cost and fitting advantage compared prior devices. Fitting of a pylon and foot still requires skill and can be expensive.

### SUMMARY

An embodiment provides a unibody transtibial prosthetic device that includes a socket configured to attach to a residual limb. A pylon extends from the socket, the pylon being a unitary polymer structure of interconnected elongated supports having open spaces therebetween. The device also includes a foot-ankle complex, the foot-ankle complex being a unitary polymer extending from the pylon, the foot and ankle unitary structure being shaped to provide multi-axial dynamic flex.

An embodiment provides a transtibial prosthetic device comprising a pylon configured as a unitary truss structure formed of a plurality of elongated supports interconnected at nodes and having open spaces between supports of the plurality of elongated supports. The unitary truss structure comprises a polymer. A foot-ankle complex extends from a base of the pylon. The foot-ankle complex comprises a sole portion; an s-shaped posterior portion extending from a first location on the sole portion to the base of the pylon; and an s-shaped anterior portion extending from a second location on the sole portion to a terminal portion of the s-shaped anterior portion near the base of the pylon. The terminal portion is unconnected to the base of the pylon. The s-shaped posterior portion and the s-shaped anterior portion are separated from each other by a gap that extends from the terminal portion to the sole portion.

An embodiment provides a transtibial prosthetic device comprising a pylon configured as a unitary truss structure formed of a plurality of elongated supports interconnected at nodes and having open spaces between supports of the plurality of elongated supports. The unitary truss structure has a non-repeating geometry, and at least some of the supports of the plurality of elongated supports are oriented diagonally with respect to a central longitudinal axis of the pylon and curve three-dimensionally around the central longitudinal axis. A foot-ankle complex extends as a unitary single piece from a base of the pylon, the foot-ankle complex comprising an s-shaped posterior portion, an s-shaped anterior portion, and a sole portion. The s-shaped posterior portion and the s-shaped anterior portion both extend from the sole portion, have upper portions with curvatures that are concentric with each other, and are separated from each other by a gap. A terminal portion of the s-shaped anterior portion is near the base of the pylon and is unconnected to the base of the pylon.

Methods for producing a transtibial prosthetic device include generating a scan of a reference prosthetic limb, wherein the reference prosthetic limb has a reference socket, a reference pylon, and a reference foot; and generating a digital model for the transtibial prosthetic device. The transtibial prosthetic device comprises i) a socket, ii) a pylon comprising a unitary truss structure, and iii) a foot-ankle complex, wherein an alignment of the pylon with the socket and with the foot-ankle complex is based on the scan. The methods also include fabricating, using the digital model and 3D printing, the pylon and the foot-ankle complex as a unitary single piece.

Methods for producing a unibody transtibial prosthetic device include acquiring patient data via imaging and/or scanning; constructing a 3D model from the patient data; translating the 3D model to 3D printable design of a unibody transtibial prosthesis; and 3D printing the unibody transtibial prosthesis. The unibody transtibial prosthesis comprises a pylon and foot-ankle complex. The pylon has a unitary truss structure formed of a plurality of elongated supports interconnected at nodes. The foot-ankle complex extends from a base of the pylon and comprises: i) a sole portion; ii) an s-shaped posterior portion extending from a first location on the sole portion to the base of the pylon; and iii) an s-shaped anterior portion extending from a second location on the sole portion to a terminal portion of the s-shaped anterior portion near the base of the pylon, the terminal portion being unconnected to the base of the pylon.

### BRIEF DESCRIPTION OF THE DRAWINGS

FIGS. 1A-1B are perspective views of an example of a unibody transtibial prosthetic device, in accordance with some aspects.

FIGS. 1C-1E are views of another example of a unibody transtibial prosthetic device, in accordance with some aspects.

FIG. 1F is a flowchart of a workflow for providing a custom fit unibody transtibial prosthetic device to a patient via imaging and 3D printing, in accordance with some aspects.

FIG. 2 shows a process for generation of a patient fit socket portion, in accordance with some aspects.

FIGS. 3A-3C illustrate, respectively, a 3D scan of a tibial region of a contralateral healthy leg, topology optimization of pylon region and a smooth version of the optimized pylon, in accordance with some aspects.

FIGS. 4A-4C illustrate respective forefoot, heel, and u-axis loading conditions, in accordance with some aspects.

FIGS. 5A-5B illustrate a process for the design of a unibody transtibial prosthetic device based upon a patient's contralateral healthy limb, in accordance with some aspects.

FIG. 6 illustrates a multi-material foot/ankle complex, in accordance with some aspects.

FIG. 7 illustrates a multi-material unibody transtibial prosthetic device with a modified socket, in accordance with some aspects.

FIG. 8 is a flowchart of a method of producing a transtibial prosthetic limb using reference prosthetic device, in accordance with some aspects.

FIGS. 9A-9C are views of a reference prosthetic limb, in accordance with some aspects.

FIGS. 10A-10B illustrate taking measurements of a reference prosthetic limb, in accordance with some aspects.

FIGS. 11A-11D show examples of reference markers, in accordance with some aspects.

FIGS. 12A-12C show how a scan of a reference socket is aligned with a reference prosthetic limb, in accordance with some aspects.

FIGS. 13A-13C illustrate an example of aligning a reference socket and a reference prosthetic limb without use of reference markers, in accordance with some aspects.

FIGS. 14A-14B show views of a reference prosthesis scan, in accordance with some aspects.

FIGS. 15A-15B show views of a digital model of a transtibial prosthetic limb, in accordance with some aspects.

FIGS. 16A-16C show views of generating a digital model, in accordance with some aspects.

FIGS. 17A-17C show views of a fabricated transtibial prosthetic device, in accordance with some aspects.

FIGS. 18A-18C illustrate a pin-lock suspension component for a socket, in accordance with some aspects.

FIG. 19 is a simplified schematic diagram showing an example computer system for use in the methods and systems of the present disclosure.

### DETAILED DESCRIPTION

Unibody transtibial prosthetic devices are described that provide an endoskeletal unibody design, bioinspired aspects, and foot design that provides a smooth, symmetrical gait and energy capture and return using a purely passive structure and without sliding connections. The endoskeletal unibody design includes a pylon having a truss structure of interconnected elongated supports. The pylon is shaped to provide multiaxial flexibility and vertical loading and displacement. The term “endoskeletal” as used herein refers to a unitary truss structure that is configured to enable adjustment of alignment, where the adjustment in the present disclosure can be achieved by thermoforming the truss structure after the 3D printing process.

Also described are patient-specific design workflows. The workflows include a series of digital scanning, design, and fabrication steps that enable many benefits that include a lower cost, more lightweight (resulting in better comfort and reduced fatigue), higher precision (resulting in a comfortable fit), and more robust (e.g., durable and resistant to environmental elements such as water, sand and mud) prosthesis to be provided compared to conventional molding, sculpting, and assembly processes. Some aspects include a 3D printed prosthetic limb with a custom fit socket fabricated from a digital model of the residual limb. Some aspects include modeling a 3D printed prosthetic limb using characteristics such as alignments and sizing from a patient's existing prosthetic limb (i.e., a “reference” prosthetic limb/device or “initial” prosthetic limb/device) and fabricating a new, customized prosthetic limb from the model as a one-piece device that includes the endoskeletal unibody design with the bioinspired truss structure. The new prosthetic with the unitary truss structure can provide improved durability and performance compared to the patient's previous (existing) prosthetic limb and can also be customized for certain situations (e.g., tailored for certain sports or recreational activities performed by the patient).

Aspects of the present disclosure uniquely enable digitally designing and printing the unitary pylon and ankle-foot structures described herein (optionally being unitary with a socket as well) that capture the fit and alignment of a patient's existing prosthetic limb. In contrast, all conventional prostheses must be dynamically aligned by a skilled prosthetist with a patient. Methods of the present disclosure advantageously enable a “ready to walk” prosthesis off the printer that is dynamically aligned (i.e., customized, tailored for an individual patient) without the need for fitting by a prosthetist. As a result, the customized prostheses that are based on an existing prosthetic limb are more accessible to patients than conventional prosthetic limbs and may save time and cost as well.

Aspects of the design workflows include digital modularity, where the design is modular in the digital assembly workspace but printed in a unitary single piece. Benefits of this modularity include an ability to utilize a saved digital assembly workspace to swap out a component—such as the socket if the patient's residuum has changed—and then recombine the component into the prosthesis with the exact same saved alignment.

Various examples of the disclosed prosthetic devices and methods of fabricating the devices will now be discussed with respect to drawings and experiments. Broader aspects will be understood by artisans in view of the general knowledge in the art and the description of the experiments that follows.

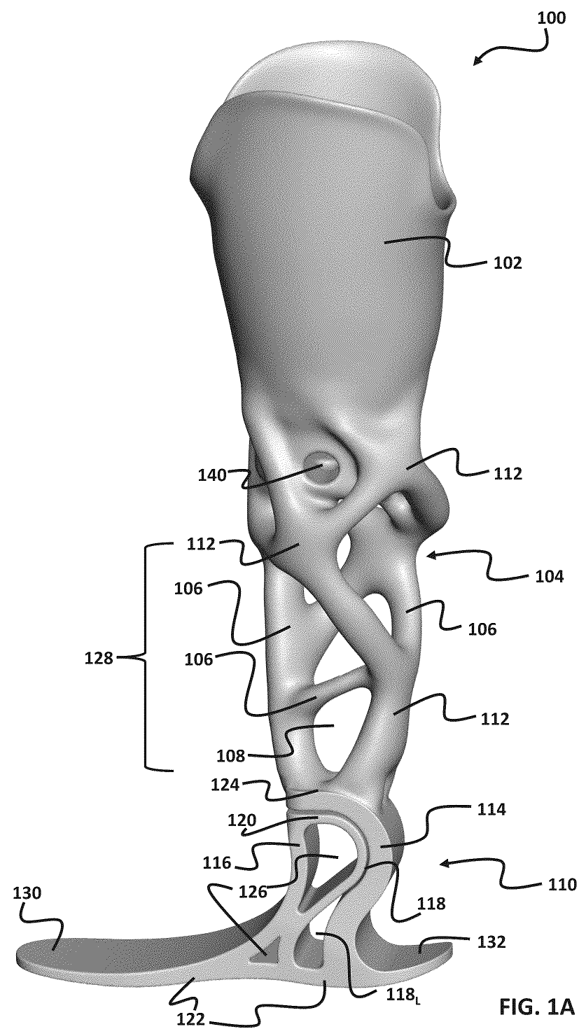

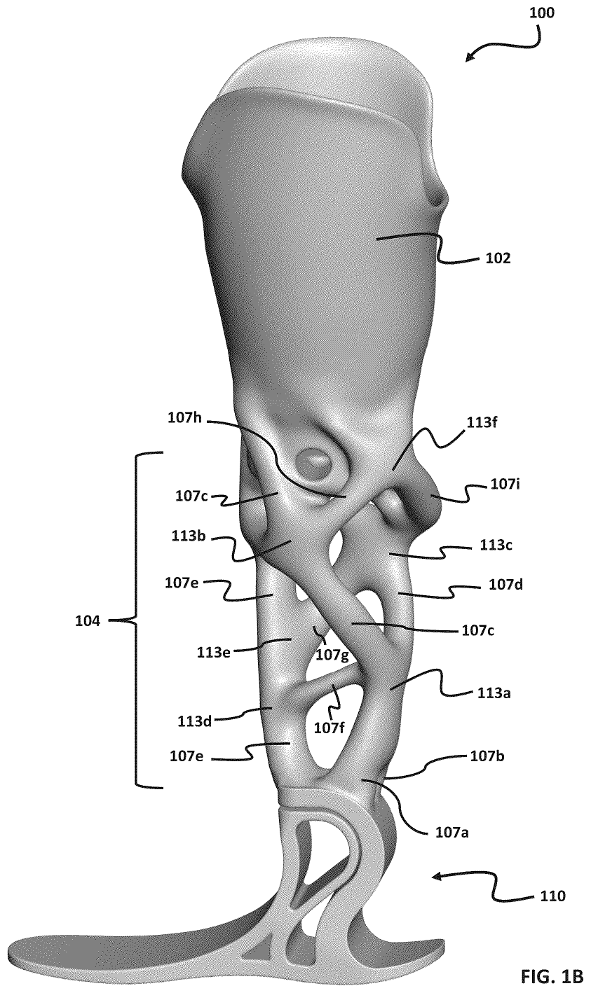

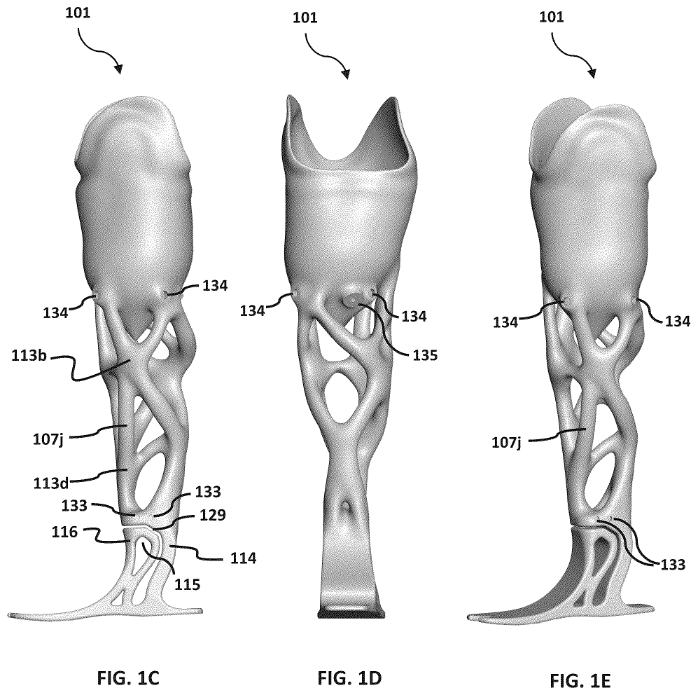

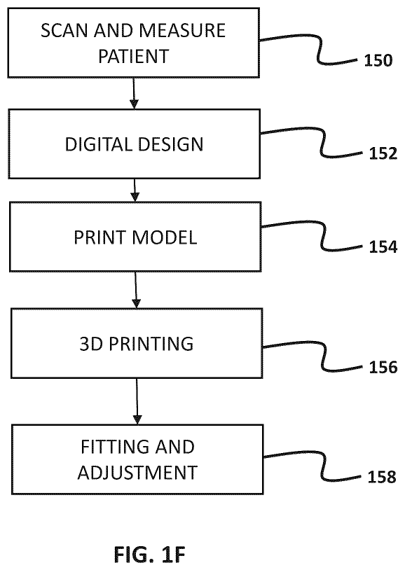

FIG. 1A shows an example of a unibody transtibial prosthetic device 100, with the medial side of the device 100 in front. The prosthetic device 100 includes a socket 102 configured to attach to a residual limb. A pylon 104 extends from the socket 102. The pylon 104 is a unitary bioinspired truss structure of interconnected elongated supports 106 having open spaces 108 therebetween. The bioinspired truss structure is based on a cholla cactus, where the woody-skeleton of the cactus generally has a central open cavity and diagonally curving struts that intersect each other at angles and form openings between them. The pylon 104 is made from thermoplastic material, in some examples. A foot-ankle complex 110 is a unitary polymer extending from a base (i.e., lower or distal portion) of the pylon 104. The foot-ankle complex 110 is a unitary structure and is shaped to provide multi-axial dynamic flex to enable dorsiflexion, plantar flexion, inversion and eversion movement for smooth symmetric gait performance.

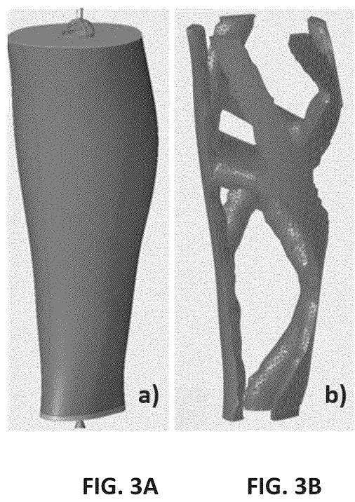

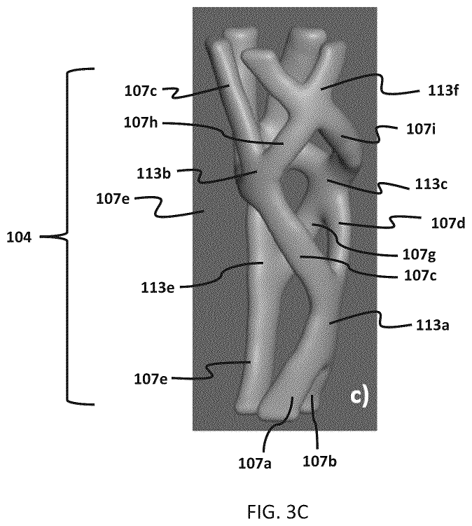

In some examples, the socket 102, pylon 104 and foot-ankle complex 110 are formed as a unitary single piece. Single and multiple materials with different properties in different regions can be used to print a unitary single piece with tailored stiffnesses in different regions. Through the combination of 3D scanning and topology optimization, the pylon 104 can be shaped and dimensioned to be within extended external boundaries of the residual limb and can be very lightweight with the total amount of polymer material in the pylon 104 being minimized to achieve a given targeted K-level of use. Further, the truss structure formed by interconnected supports and nodes and blended with the ankle-foot complex provides a dynamic, multiaxial response during the gait cycle with energy capture and release achieved through the entire shin-ankle-foot. The optimization process for the pylon 104 includes the example discussed below with respect to FIGS. 3A-3C.

The entire prosthesis can be very lightweight but still be for a K-level 2 through K-level 4. As seen in FIG. 1A, the interconnected elongated supports 106 have connection node portions (“nodes”) 112 and define a central open cavity and curve around the open cavity. That is, the interconnected elongated supports 106 gently curve or arc into the nodes 112 to connect at the nodes 112, and a cavity exists between the interconnected elongated supports 106 along a central longitudinal axis of the pylon 104. At least some of the interconnected elongated supports of the unitary truss structure are oriented diagonally with respect to a central longitudinal axis of the pylon and curve three-dimensionally around the central longitudinal axis. The unitary truss structure with elongated supports joined at nodes and forming a hollow (central open cavity) and generally cylindrical framework was uniquely inspired by cholla wood as described above. This bioinspired structure provides strength for withstanding compressive and torque loads while also allowing individual supports to be adjusted for customized alignment of a specific patient. For example, after the pylon is fabricated, thermoforming may be used to make alignment adjustments to the prosthesis, which may include rotating, stretching, and/or bending the pylon structure. Overall alignment adjustments to the prosthesis include rotational, angular and slide (translational) adjustments.

The central open cavity has a decreasing diameter from the socket 102 to the foot-ankle complex 110 in a manner resembling the decreasing size of a human transtibial leg portion. An outer imaginary boundary of the interconnected elongated supports and nodes 112 may follow the mirrored shape of the contralateral transtibial leg portion of a patient, in some examples. With imaging and 3D printing methods of making discussed below, the mirrored model is based upon the 3D scan data from the contralateral transtibial leg portion of a patient, if available. If not available, then another embodiment is a pylon structure that uses starting geometry of a transtibial leg portion from a database. The database model can be selected based on weight, activity level and aesthetics, among other things, and will be scaled to appropriately fit the specific patient. Generally, the interconnected elongated supports 106 and open spaces 108 can define a bioinspired truss structure with an outer boundary that follows the mirrored model of the sound contralateral leg of the patient. In other examples, the outer imaginary boundary and design of the interconnected elongated supports and nodes 112 may be based on the shape of an existing prosthetic limb that the patient already has, as described later in this disclosure.

In the example of FIG. 1A, the unitary truss structure may have a non-repeating geometry, where the orientation and connecting of elongated supports do not form a repeating pattern over the length of the pylon 104. An example configuration of interconnected elongated supports is described in FIG. 1B, where supports 106 of FIG. 1A shall be referred to individually as supports 107n, and nodes 112 of FIG. 1A shall be referred to individually as nodes 113n. Some of the annotations of FIG. 1B are also shown in FIG. 3C for clarity.

In FIG. 1B, two supports 107a and 107b extend upward from a distal end of the pylon 104 (where the pylon 104 joins the foot-ankle complex 110), to connect to each other in an upside-down V-shape at a node 113a. From node 113a, two supports 107c and 107d angle upwards in a V-shape, curving three-dimensionally and diagonally around the tapered cylindrical shape of the pylon 104. Support 107c is on a medial side of pylon 104, intersects a node 113b, and may continue proximally to socket 102. Support 107d runs toward the lateral side of pylon 104, intersects a node 113c, and may also continue proximally to socket 102.

On the anterior and distal end of pylon 104, a support 107e extends from the foot-ankle complex 110 upward toward the socket 102. At a node 113d along support 107e, a support 107f may extend diagonally in an approximately anterior-posterior direction between node 113d and node 113a. At a node 113e along support 107e, above (proximal to) node 113d, a support 107g may extend between node 113e and node 113c, curving three-dimensionally upward from anterior to posterior. A support 107h extending diagonally upward and posterior from node 113b may intersect a node 113f at a medial-posterior location near the socket 102. A support 107i extending diagonally upward and in an anterior direction from node 113c may also intersect 113f.

Nodes 113a and 113d are toward the foot-ankle complex (e.g., in a distal half of the pylon 104). Node 113e is proximal to node 113d in this example (i.e., more toward the socket 102 than node 113d), in a central portion of pylon 104. Nodes 113b, 113c and 113f are near the socket 102, with node 113f being more proximal (i.e., more toward the socket 102) than nodes 113b and 113c) in this example.

As can be seen in FIGS. 1A and 3C, the bioinspired (cholla-like) truss structure incorporates elongated supports that form angled, X-shaped intersections, where the supports form an open framework in the shape of a patient's calf, around a central open cavity. For example, supports 107a, 107b, 107c and 107d form an X-shape around node 113a (see FIG. 3A). X-shaped intersections are also formed at, for example, nodes 113b, 113c, and 113f. Other nodes such as nodes 113d and 113e may have supports intersecting in other angular formations such as Y-shaped, K-shaped or V-shaped. This network of interconnected supports and nodes, that may form diamond-shaped or triangular openings uniquely provides strength that can withstand multi-axial compression and torque forces. The result is an endoskeletal design in which the positions and angles of the supports and nodes can be adjusted by thermoforming to customize the pylon for an individual patient. Furthermore, the lengths and thicknesses (or cross-sectional shape) of the supports may be customized according to the patient's needs such as their height, weight, activity level and gait.

The example material for the pylon 104 and for the entire prosthesis 100 is a 3D printable thermoplastic. With regard to the pylon 104, the thermoplastic material permits alignment adjustments in the heat zone 128 during a patient fitting, if necessary. In addition, the fit of the socket can be modified via thermoforming any specified region where pressure needs to be increased or decreased. However, it is desirable that the prosthesis is aligned properly during the design phase and does not require any modifications post-fabrication, and present methods provide a better chance of a patient specific prosthetic that can be used without any post-fabrication methods. While the leg can very closely model a missing leg of a patient, it will be the rare case where some adjustment is not needed. Advantageously, due to the endoskeletal design, by heating the pylon to a pliable temperature, adjustments can be made during a final fitting of a patient.

The foot-ankle complex 110 includes a sole portion 122; an s-shaped posterior portion 114 extending from a first location on the sole portion 122 to the base of the pylon; and an s-shaped anterior portion extending from a second location on the sole portion to a terminal portion of the s-shaped anterior portion near the base of the pylon, the terminal portion being unconnected to the base of the pylon. The s-shaped posterior portion 114 and s-shaped anterior portion 116 are separated from each other by a gap 118 extending from a terminal portion 120 of the s-shaped anterior portion 116 to a sole portion 122. This defines a split ankle portion that gives a dynamic response with energy recapture. The gap is preferably small in its upper portion and widens to a teardrop profile shape in its lower portion 118L. The gap is located below the pylon and extends downward to a sole of the foot-ankle complex, where in some examples an upper portion of the s-shaped posterior portion and an upper portion of the s-shaped anterior portion have curvatures that are concentric with each other. In some examples, near the terminal portion of the s-shaped anterior portion, the gap runs transverse to the central longitudinal axis of the pylon and along the base of the pylon. In some examples, the terminal portion of the s-shaped anterior portion is directly below an anterior end of the base. In some examples, the gap is located within a volume below the base of the pylon, the volume having a cross-sectional area of the base of the pylon.

An upper portion 124 of the posterior s-shaped portion 114 can be unitary with a lowest portion of the pylon 104, curving toward the central longitudinal axis and blending into the pylon, and essentially forming a base of the pylon 104 with the pylon 104 and foot-ankle complex 110 forming a unitary single piece structure with each other and also with the socket 102. To handle more force through the pylon 104 and foot-ankle complex 110, the s-shaped posterior portion 114 can be solid and substantially thicker than the s-shaped anterior portion 116, which itself can include one or more pass-through openings 126 within the s-shaped anterior portion 116. The sole portion 122 may include a split toe 130 and a split heel 132. The split toe and heel are useful for uneven terrain, where exaggerated inversion and eversion is beneficial. Overall, the unitary foot and pylon portions have no mechanical linkages, joints or sliding mechanisms, such as ball-socket connections or multi-link pinned connections, which will result in long-term durability.

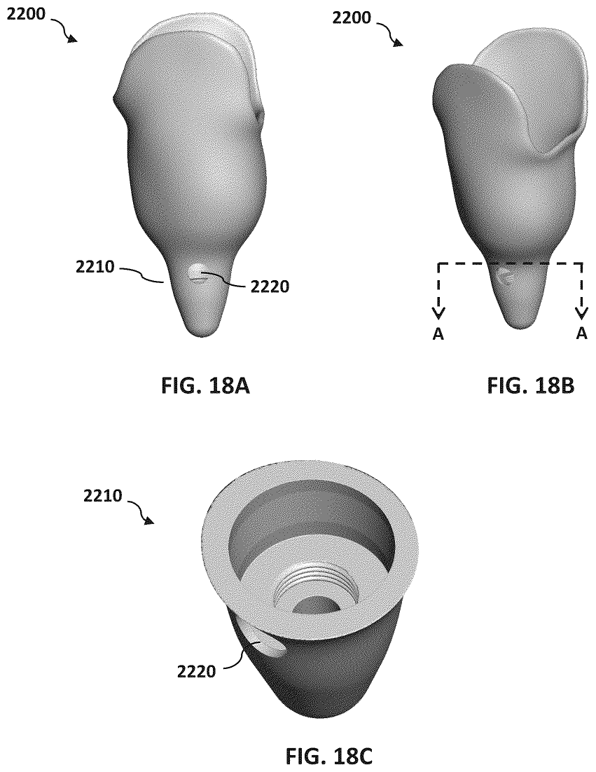

FIGS. 1C-1E are medial, posterior and perspective views, respectively, of another example of a unibody transtibial prosthetic device 101, in accordance with some aspects. The truss structure of unibody transtibial prosthetic device 101 is similar to FIGS. 1A-1B, but with an additional support 107j that extends from node 113d and curves diagonally from anterior to medial to node 113b. In unibody transtibial prosthetic device 101, the ankle has a 45-degree angled section 129 in the concentric areas of the s-shaped posterior portion 114 and s-shaped anterior portion 116 to improve printability compared to rounded shapes, and two 45-degree angles in the upper interior area 115 of the ‘D’ to improve printability. Datums (as shall be described later in this disclosure) are also included in the unibody transtibial prosthetic device 101. Datums 133 in the region of the distal pylon connection to ankle are configured as circular indentations printed into the device in this example. Four datums are included in this ankle area, two on the medial side as shown and two on the opposite side (lateral side, not visible in these views). Four datums 134 are also included the proximal pylon connection to the socket, configured as circular indentations printed into the device in this example. The posterior view of FIG. 1D shows a suspension component mating feature 135, which shall be described in relation to FIGS. 18A-18C. The suspension component mating feature 135 may be adaptable with, for example, an expulsion valve. After printing the unibody transtibial prosthetic device 101, the suspension component mating feature 135 is thread-tapped to enable the expulsion valve to screw onto the socket and seal against the flat face of the suspension component mating feature 135.

The prosthesis can meet requirements for government run health systems or insurance carriers. As an example, the United States Medicare program uses “L Codes, such as the following examples.

L5301—below knee, molded socket, shin, SACH foot, endoskeletal system (base code). The present prosthesis 100 meets this and L5637—addition to lower extremity, below knee, total contact socket has total contact surface against residual limb.

L5645—addition to lower extremity, below knee, flexible inner socket, external frame inner socket pockets that add flexibility and increase comfort, prominent bone projection can be met with tailored soft areas in the socket.

L5647—addition to lower extremity, below knee suction socket. The socket can include a one-way expulsion valve with a suspension sleeve to create a suction fit.

L5671—addition to lower extremity, below knee/above knee suspension locking mechanism (shuttle, lanyard or equal), excludes socket insert pin lock. Generally, the socket can be fabricated to work with any conventional attachment scheme, including suspensions.

L5940—addition, endoskeletal system, below knee, ultra-light material (titanium, carbon fiber or equal). The present pylon and foot/ankle structure can be very lightweight. The present socket, pylon, and/or foot-ankle can be made from ultra-light composites such as carbon fiber reinforced thermoplastic. The open endoskeleton style components minimize material used, and can also use very light weight. In addition, the use of a single unitary on-piece design can eliminate metals used in conventional pylons and attachments between components.

L5910—addition, endoskeletal system, below knee, alignable system. As described below, the prosthesis of the present disclosure can be adjusted via a thermoforming alignment process.

L5981—all lower extremity prostheses, flex-walk system or equal dynamic response integrated pylon foot. The present unitary prosthesis meets this code.

For a patient-specific fitting and fabrication method, an example method is illustrated in FIG. 1F. Patient image acquisition (block 150, scan and measure patient) can include photos, scan data or both, with the purpose of creating a digital twin of the patient. Photos can even be taken from a smart phone or other mobile device (e.g., a computer tablet), which was demonstrated experimentally in the successful fabrication of a patient-specific prosthesis. A 3D model is next created in block 152 (digital design) from the images and/or scan data. In one example, modelling is conducted from the photo data using structure from motion (SfM) techniques or with a structured light software (SL). For SfM, see C. Bregler, A. Hertzmann, and H. Biermann, “Recovering non-rigid 3d shape from image streams,” Proceedings IEEE Conference on Computer Vision and Pattern Recognition. CVPR 2000 (Cat. No. PR00662); F. Dellaert, S. Seitz, C. Thorpe, and S. Thrun, “Structure from motion without correspondence,” Proceedings IEEE Conference on Computer Vision and Pattern Recognition. CVPR 2000 (Cat. No. PR00662). For SL, J. Geng, “Structured-light 3d surface imaging: a tutorial,” Advances in Optics and Photonics, vol. 3, no. 2, p. 128, 2011; S. Dunn, R. Keizer, and J. Yu, “Measuring the area and volume of the human body with structured light,” IEEE Transactions on Systems, Man, and Cybernetics, vol. 19, no. 6, p. 1350-1364, 1989. In another example, a smartphone with a facial recognition camera, such as the Apple® iPhone® XR TrueDepth® Camera, is used to scan the patient using an app such as COMB. This enables a low-cost, highly mobile solution to produce a digital twin of the entire patient in STL format. In another embodiment, the facial recognition camera is used to capture only the regions of interest, such as amputated limb and foot and calf sections of their contralateral limb. In another embodiment a structured light scanner is used to scan either the entire patient or specific regions of interest necessary to design the unibody prosthetic limb. In another embodiment, a series of photographs are taken using any digital camera, such as the rear facing camera on any smartphone and reconstructed using the photogrammetry technique to create a point-cloud digital twin of the patient.

G-code for printing (print model) is generated in block 154 from the 3D model. This is accomplished via slicing software. Example settings used in experiments included a nozzle size 1.4 mm, layer height 0.64 mm, print speed 2000 mm per minute, nozzle temperature (varies by material between 240-280 degrees Celsius), bed temperature (varies by material between 90-140 degrees Celsius), chamber temperature (varies by material between 70-90 degrees Celsius), infill 100% density. These settings are used in the slicing software, regardless of which software is used. These settings control how the printer behaves during manufacturing. Artisans will recognize adjustments based upon materials being printed. The prosthetic is then printed via 3D printing in block 156, which can be printing remotely and shipping to a fit practitioner/patient or sending print data for printing near the fit practitioner/patient. This allows a patient to be imaged where they reside and also receive a prosthesis at that location.

Fitting (e.g., testing) and adjustment in block 158 includes the patient testing the prosthetic for fit and performance. A fitting practitioner can make minor adjustments to alignment and socket fit through thermoforming. In one example, a heat gun is used to raise the temperature of the heat zone 128 until it is pliable (above the material's glass transition temperature and below the material's melting temperature, checked using a handheld laser temperature gauge). In another example, radiative heating is used to raise the temperature of the heat zone 128 until it is pliable. Other methods such as using a torch/flame may lead to difficulty controlling heat distribution but are still possible methods for heating the heat zone 128. In some cases, alignment is controlled using an alignment fixture that would clamp the ankle in place and allow for controlled rotation and translation in the sagittal, frontal, and transverse planes. Rotation should be controlled to less than one degree and translation precision to one millimeter. The socket is heated locally to adjust fit in a specific area where pressure may need to be relieved or increased against the patient's amputated limb. Adjustments to fit within the socket are achieved manually using a sturdy wooden bar with a rounded end that is pressed into the region of interest to increase or relieve pressure on the patient's amputated limb. As a note, thermoforming is a common practice in traditional prosthetic clinics as many check sockets, socket liners, etc. are thermoplastics. However, traditional prosthetic sockets often employ composite materials so fit cannot be adjusted. This gives us an advantage because we can easily modify our socket if a patient experiences discomfort either during initial fitting or during a follow up appointment during the device lifetime. The general design of the foot can be standard, i.e. have the S shaped posterior and anterior sections, the split design starting narrow and going to a teardrop shape. However, the actual dimensions of the foot are customized for every patient based on their weight, activity level, use-case, etc.

The FIG. 1F method permits very convenient service and business models. For example, a patient can receive a functional prosthetic without ever physically meeting with a traditional specialist. In areas where no skilled prosthetists are available, a non-specialized practitioner should be able to make minor adjustments to the printed limb via the thermoforming process. It is always preferred to have a licensed prosthetist do the fitting and walking test, but in many parts of the world this is not feasible, and a prosthesis of the present disclosure provides a workable alternative in such situations.

In some fitting and fabrication methods of this disclosure, imaging of a patient can be conducted remotely, and the data can be sent to the designer and 3D printing facility. Image data of a contralateral limb and portions of a residual limb are used with an example method implemented in software to create an optimized patient-specific printed prosthesis in accordance with FIG. 1A. Additional features of the method of FIG. 1F will be understood by artisans with reference to the following discussion of experiments that show imaging, printing, fitting and testing of prototype prostheses described herein.

3D printing can be generally considered an additive fabrication process. Other additive processes can be used. Additive processes should be able to deposit thermoformable materials and preferably allow more than one material in a single deposition, e.g., material extrusion, powder bed fusion and material jetting techniques. Material extrusion may be one method used as a fabrication technique, such as fused filament fabrication (FFF).

The prosthesis can be made from various materials, such as a thermoplastic which is beneficial for adjustment. Typical polymers used for 3D printing are; ABS, PLA, PC, PETG, PA, PEI and PEEK. Generally, any material that can be deposited and solidified can be used to form a prosthesis by the present methods. Example materials include thermoplastic pellets, filaments, and resins, and may contain additives such as foaming agents, fibers, and particles. Relevant processing temperatures range from 180-500 degrees Celsius. The material must adhere to a previous layer with a sufficient bond strength and should be reformable through the application of heat. The selected material preferably exhibits high elongation and excellent interlayer bonding. High elongation means an ultimate elongation of greater than 150%. Excellent interlayer bonding means high interlayer bonding leading to a tensile strength perpendicular to layers no less than 60% of the tensile strength parallel to layers of the material.

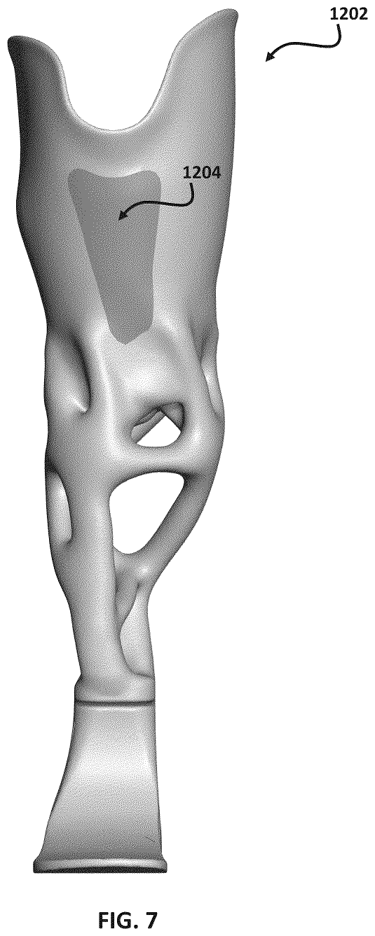

The 3D printing of block 156 can advantageously be multi-material printing to produce a unitary single-piece device. Use of multiple materials allows tailoring stiffness locally in the single print, which can improve gait performance, comfort, strength. For example, sensitive contact areas of the socket (see FIG. 7, area 1204) can be made softer than other regions to improve comfort. Another example includes a core-shell pattern where a 1-5 mm shell of the outer surface of the entire unibody (or any of the three portions) is flexible material and the core material is stiffer. Materials such as thermoplastic elastomers, thermoplastic urethanes, unfilled nylons and polypropylene, among others, would act as the flexible material, where materials such as chopped carbon or glass fiber filled nylons or polypropylenes, polycarbonate, PEEK, and PEI, among others, could act as the stiff material.

### Experimental Data

Experiments demonstrated cost-effective 3D modeling of a patient's damaged limb through the use of smartphones and photogrammetry techniques. Some testing used separate components as a precursor to unitary single-piece transtibial prosthesis.

### Patient Specific Workflow

Methods for producing a unibody transtibial prosthetic device may involve acquiring patient data via imaging and/or scanning; constructing a 3D model from the patient data; translating the 3D model to 3D printable design of a unibody transtibial prosthesis; and 3D printing the unibody transtibial prosthesis. An example workflow starts with a scan of amputee via the Comb scanner app (Comb O&P, Chardon, OH) which uses the facial-recognition camera in smartphones to generate 3D models of the patient. Scan data is supplemented by a patient measurement form and used to design a personalized prosthetic limb that follows the external shape of their mirrored contralateral limb. The digital design process for every device is done under the supervision of a certified prosthetist (CPO). The socket may be created using scan data and patient measurements with prosthetic design software Neo (Rodin4D, Italy). The pylon with its present bioinspired truss structure may be created using topology optimization software nTopology (nTopology, New York, NY) controlled to implement a pylon of the present disclosure having a unitary bioinspired truss structure of interconnected elongated supports. The multi-axial dynamic foot-ankle complex may be created in Fusion360 (Autodesk, San Rafael, CA) controlled to implement a foot-ankle complex of the present disclosure that provides multi-axial dynamic flex. Finally, alignment and blending of the socket, pylon, and foot-ankle complex into the present unitary single-piece transtibial prosthesis may be done in Meshmixer (Autodesk, San Rafael, CA). The finished model may be sliced into G-code that the 3D printer can interpret using Simplify3D (Simplify3D, Cincinnati, OH). The fused filament fabrication (FFF) manufacturing process uses engineering grade thermoplastics to produce a strong and durable endoskeletal prosthetic device. The UniLeg is shipped to a prosthetist or physician who helps the amputee put on their personalized limb and confirms a smooth, symmetric gait. If any modifications are desired, thermoforming can be used to adjust fit and alignment.

### Imaging and Modelling

Pervasively available smartphone imaging technology is useful to create 3D models of the patient. The Comb scanner app (Comb O&P, Chardon, OH) uses the front-facing facial-recognition camera available on many smartphones, e.g., the TrueDepth camera on iPhones (Apple, Cupertino, CA). Imaging can be performed on-site anywhere in the world and the scan data and measurement form are then sent to a fabricator (or a cloud operated by the fabricator). At a minimum, scans are taken of the amputee's residual limb and contralateral side.

### Socket Design

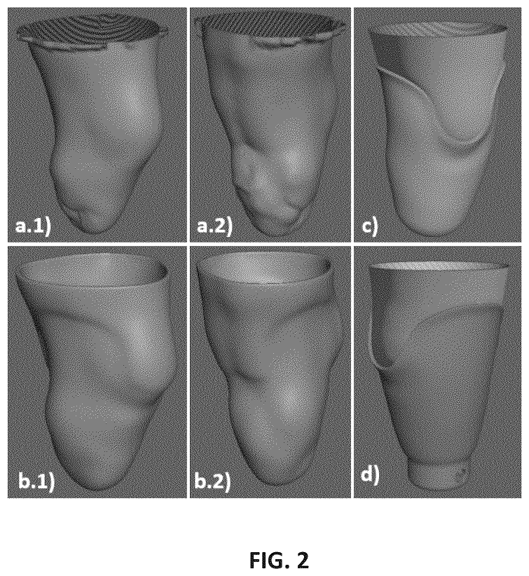

Scan data from the amputee's residual limb is used as the starting shape for the socket, in a process illustrated in FIG. 2. Modifications are made with Neo (Rodin4D, Italy) to properly distribute load and prevent pressure spots. The process is as follows: (1) the mesh is cleaned, smoothed, and the volume is reduced by 3% (a1,2); (2) a 2 cm wide by 1 cm deep slot is carved for the patellar tendon bar, which is a primary load-bearing feature, and the medial-lateral area is compressed by 2% (b1,2); (3) socket trim lines are delineated to allow proper knee mobility and the socket walls are thickened to 4 mm (c); (4) an extruded section at the distal end is created to house the shuttle-lock (d). Modification values can change from patient to patient, and this process can benefit from guidance by a CPO.

### Pylon Design

A pylon of the prostheses described herein is designed to be strong and 3D printable, while reducing material use to reduce cost and manufacturing time. The pylon is expected and configured to withstand a series of compressive and torque loads based on patient weight and activity level and following ISO standard 10328. Topology optimization (TO) software nTopology (nTopology, New York, NY) is used to generate an optimal truss structure consistent to provide the unitary bioinspired truss structure of interconnected elongated supports having open spaces therebetween. The mirrored model of the transtibial section of the amputee's contralateral limb is used to generate the pylon. In cases where a bilateral amputee doesn't have a sound limb, a generic transtibial limb can be selected from a database and then reoriented and scaled to fit the patient. Model preparation for TO requires the definition of design space (FIG. 3A), where topology is optimized, and boundary condition regions (top and bottom disks in FIG. 3A), where loads and constraints are placed. Topology optimization is performed with the objective of maximizing stiffness.

The resultant mesh (FIG. 3B) was exported as an STL file and then imported into mesh modeling software Meshmixer (Autodesk, San Rafael, CA) to be smoothed and scaled to the correct dimensions. The final personalized bio-truss structure of the present disclosure, FIG. 3C, has an outer contour that follows that of the mirrored contralateral limb of the specific amputee. A significant advantage of the endoskeletal truss structure is that it can deform without buckling, which enables adjustment of alignment by thermoforming.

### Ankle-Foot Complex

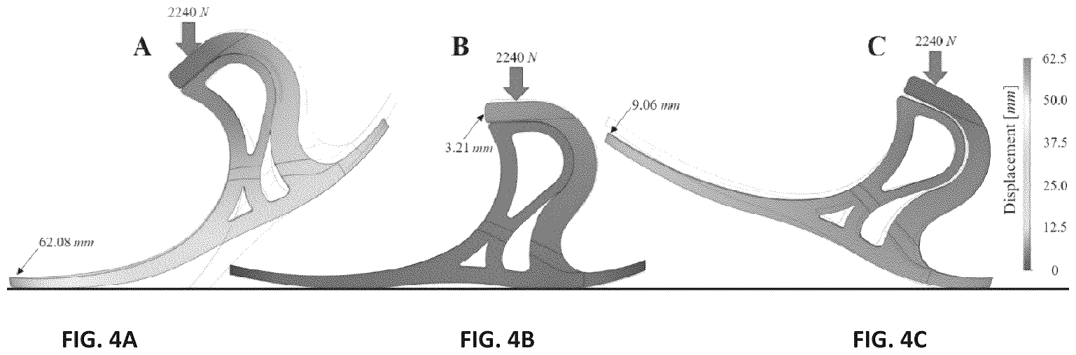

The foot-ankle complex is designed to provide a multi-axial dynamic response to enable dorsiflexion, plantar flexion, inversion and eversion motion for smooth symmetric gait performance and energy capture and return, which is illustrated in FIGS. 4A-4C. As discussed above, the foot-ankle complex includes s-shaped posterior and s-shaped anterior sections that are separated from each other by a gap. This defines a split ankle that enables plantar flexion during heel-strike, demonstrated in FIG. 4C. As the person transitions to mid-stance, FIG. 4B, the split ankle gap closes, providing stability. Transitioning to terminal stance, the entire foot-ankle complex coils to capture energy, FIG. 4A, which will be released to drive forward into the next gait cycle. The top of the ankle-foot complex is blended into the base of the pylon, which provides a smooth transfer of load from the ground through the dynamic pylon up to the socket. The sole portion may include a split toe and split heel. The split toe and heel are useful for uneven terrain, where exaggerated inversion and eversion is beneficial.

### Digital Alignment and Integration

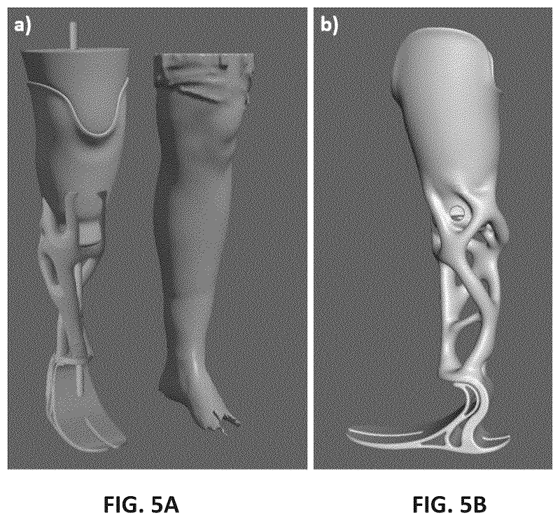

The socket, pylon, and foot-ankle complex are aligned and blended into the single-piece UniLeg in Meshmixer (Autodesk, San Rafael, CA), as illustrated FIGS. 5A and 5B. Alignment is guided using the mirrored model of the contralateral limb. A cylinder is drawn that runs through the middle of the mirrored leg to align the knee and ankle positions, FIG. 5A. The resulting unibody design, FIG. 5B, can be fully printed in one piece. An example commercial component used is a shuttle lock and pin system to secure the residual limb in the socket, such as into hole 140 of FIG. 1A. If necessary, an on-site prosthetist or physician can make minor adjustments to alignment and socket fit through thermoforming. A heat gun is used to raise the temperature of the pylon until it is pliable, which allows manual rotation and translation in the sagittal, frontal, and transverse planes. Adjustments to fit within the socket are achieved by heating the region of interest to pliability and then using a sturdy wooden bar with a rounded end that is pressed into the region of interest to increase or relieve pressure on the patient's residual limb.

Thermoforming is a common practice in traditional prosthetic clinics as many check sockets are thermoplastics. However, traditional prosthetic sockets often employ composite materials so fit cannot be adjusted. The inventive prosthesis provides a great advantage as it can be easily modified via thermoforming if a patient experiences discomfort either during initial fitting or during a follow up appointment during the device lifetime.

Validation with a Patient Fitting

A patient was fitted with a unibody transtibial prosthetic device of the present disclosure. Alignment was assessed in anterior and lateral planes with the patient standing. For proper TKA alignment, the trochanter (hip), knee, and ankle points must fall along a vertical line when standing. To quantify the benefits of the present unibody transtibial prosthetic device, a comparison of weight, cost, and time of design were made against a patient's existing prosthetic device. Weight comparison between the devices revealed a 55% weight reduction from the conventional prosthetic, which weighed 4 pounds, to the present device weighing only 1.8 lbs. Lighter weight devices reduce metabolic cost during activity and are often reported to be more comfortable. Time of design and manufacture was compared between the two design methodologies. Total traditional transtibial prosthetic leg time for design and manufacture was estimated at 14 days. This estimate includes measurements (1 Day), mold creation (3 Days), socket creation (6 Days), and assembly and alignment (4 Days). In comparison, the present unibody design and manufacture time is 16 hours, which consists of data acquisition (1 Hour), design time (3 Hours), and printing time (12 Hours).

### Modified Designs

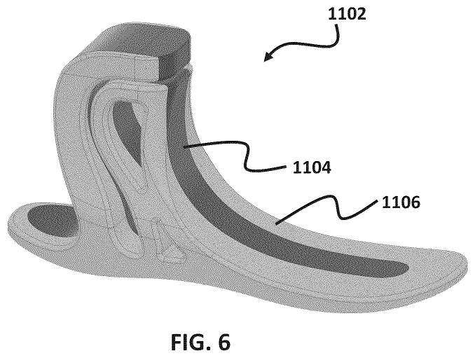

FIG. 6 shows an example configuration for foot/ankle complex 1102 that is consistent with the general shape, arrangement, and structure of the foot/ankle complex 110 of FIG. 1A. The foot/ankle complex 1102 includes central strip 1104 of stiffer material surrounded by more flexible material 1106. The ratio of the two materials controls energy return, flexibility, and dynamic range. While not shown in FIG. 6, the foot/ankle complex 1102 is unitary with a pylon section and socket section.

FIG. 7 illustrates an example unibody prosthetic 1202 of the prostheses described herein that includes one or more (interior/patient contact side) pockets (area 1204) of flexible material of pressure sensitive areas of the patient's amputated limb. These areas can be identified by the patient or physician during visit to the clinic as scanning occurs.

Patient Specific Workflow with Reference Prosthetic Limb

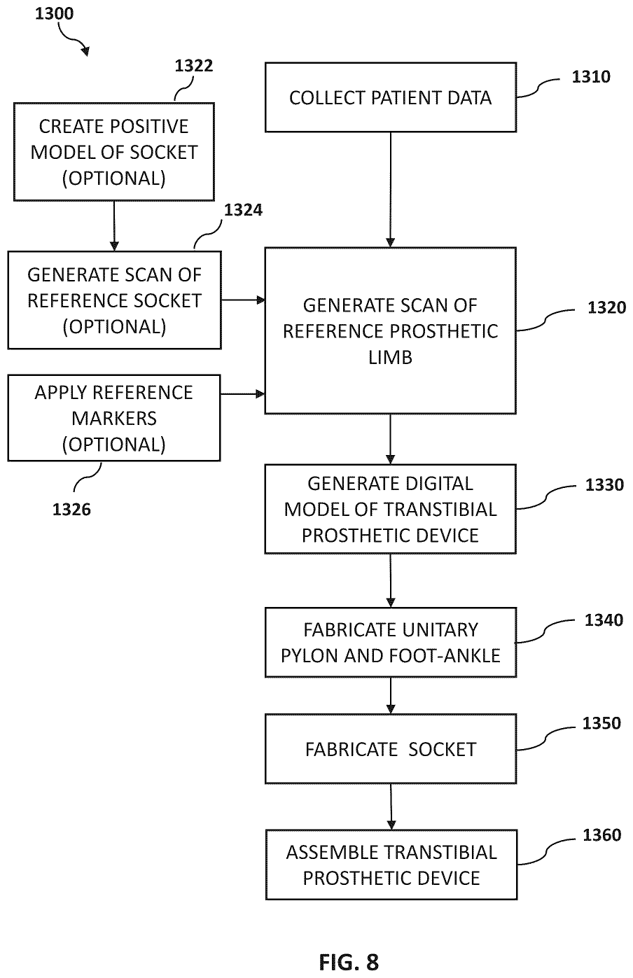

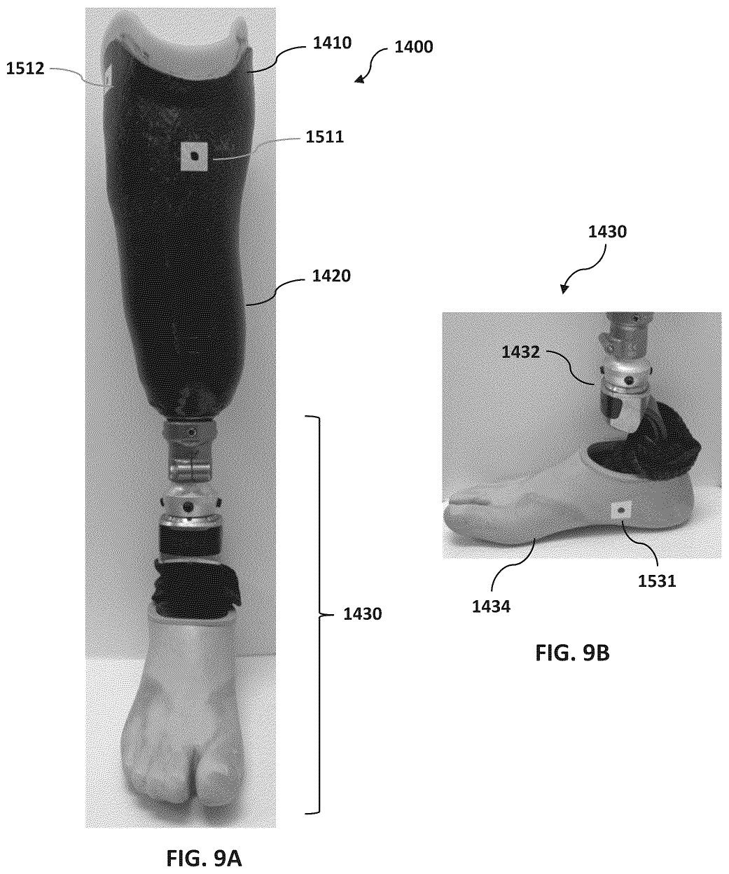

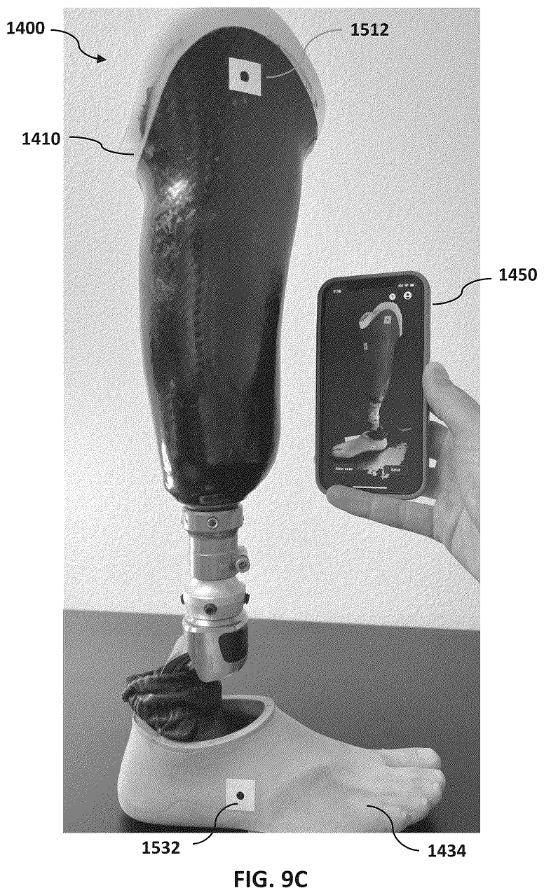

Another method of producing a transtibial prosthetic limb is described in the flowchart of FIG. 8 in which a patient's existing prosthesis is used as a basis for designing the new transtibial prosthetic limb (e.g., copying an existing prosthetic limb and adapting it to the unitary truss structure design of the present disclosure). FIGS. 9A-11D show various examples of reference prosthetic device and reference markers. In method 1300 of FIG. 8, an existing (“reference”) prosthetic device (e.g., reference prosthetic limb 1400 of FIG. 9A) that is already fitted for a patient may be scanned three-dimensionally, where the reference prosthetic device includes a reference socket, a reference pylon and a reference foot. The reference prosthetic device may be exoskeletal or endoskeletal; may be made by 3D-printing, molding, machining or other manufacturing techniques; and may be made from metal, plastic, or other materials. The reference prosthetic limb should have proper assembly and alignment for an individual patient. In some cases, the reference socket, the reference pylon, and the reference foot have been assembled and aligned by a certified prosthetist/orthotist (CPO). In these methods, the fit and alignment from the reference prosthesis can be replicated (i.e., transferred, copied) to a new UniLeg prosthesis (prosthetic device that has a unitary truss structure as described herein). In some cases, the new UniLeg prosthesis may include the unique foot-ankle complex described herein. In some cases, the new UniLeg prosthesis may include a socket fitted for the individual patient, where the socket may be a separate component from or may be integral with the pylon-ankle-foot.

In methods of using a reference prosthetic limb to design a new prosthetic limb in accordance with the present disclosure, a scan of the reference prosthetic limb is used for modeling the unibody prosthetic limb (having a unitary truss structure as described herein) rather than using a scan of the patient's contralateral limb and/or residual limb. The scan (e.g., 3D scan) beneficially provides parameters and a framework to build a properly sized and aligned prosthetic limb in a digital workspace, allowing a user to create a patient-specific prosthesis that already has been dynamically aligned for that patient before printing. As a result, little or no alignment is required after printing the new prosthetic limb. Some blocks of method 1300, such as blocks 1320 and 1330, can be performed using a computer processor and associated software (e.g., on a smartphone, computer table, desktop computer as described throughout this disclosure).

Elements of FIG. 8 are similar to FIG. 1F, but with the scan of block 150 modified for scanning an existing prosthetic limb that the patient already uses. In FIG. 8, block 1310 begins with collecting patient data, which may include entering patient information (e.g., height, weight, foot length) and taking photos of the patient wearing the reference prosthetic limb (which may also be referred to as a reference prosthesis, reference prosthetic device, initial prosthesis, initial prosthetic device, existing prosthesis, or existing prosthetic device).

A scan of the reference prosthesis is created in block 1320. That is, block 1320 involves generating a scan of a reference prosthetic limb, wherein the reference prosthetic limb has a reference socket, a reference pylon, and a reference foot. The reference prosthetic limb is prepared by positioning the prosthesis to stand vertically. A milmo may be utilized to fix the prosthesis in position. A milmo, also known as a Vertical Fabrication Jig, is used by prosthetists to position the socket and foot during fabrication and bench alignment.

Referring to reference prosthetic limb 1400 of FIG. 9A as an example, block 1320 involves scanning the socket 1410, the pylon 1420, and the foot-ankle complex 1430 of reference prosthetic limb 1400. FIG. 9A is a front view of reference prosthetic limb 1400, and FIG. 9B is a view of the medial side of the foot-ankle complex 1430 that includes reference ankle 1432 and reference foot 1434. As illustrated in FIG. 9C which is a lateral view of the reference prosthetic limb 1400, the scanning may be performed using, for example, a smartphone 1450 or computer tablet with imaging technology (e.g., a camera) as described earlier in this disclosure. The scan generated in block 1320 includes external dimensions and 3D shapes of the various portions of the reference prosthetic limb 1400. Any scanner (e.g., iPhone True Depth, scanner sensors and software applications used with an iPad®, handheld laser, handheld structured light, or other) can be used. Scanners use a wide variety of software. Depending on the type of scanner, a direct 3D model with or without color can be exported, or a point cloud may be created that is post-processed in a secondary software before using it in workflows of the present disclosure.

An internal scan of the socket 1410 is needed for fitting the patient's residual limb. For cases where a digital model of the socket is not available, method 1300 involves optional block 1322 of creating a physical positive model of the socket. A 3D scan of the positive model (using 3D scanning technology as described above for block 1320) is then scanned in block 1324 as part of generating a scan of the reference prosthesis in block 1320. That is, the generating the scan of the reference prosthetic limb may comprise scanning the physical positive model.

Various techniques may be used to generate the scan the socket of the reference prosthetic device. In one case, a positive model of the socket is created in block 1322 and then scanned in block 1324. To create a positive model, a socket duplication process may be performed. In such a case, alginate, plaster, or other material is poured into the socket 1410 to create a physical positive of the socket, which captures the internal surface of the socket. The positive is then scanned in block 1324, and then that scan is used to create a digital model of the socket for the transtibial prosthetic device in block 1330. In a second case, an existing physical positive model may be available from when the reference prosthesis was made. In this case, block 1322 is not needed, and the existing physical positive model is scanned in block 1324. In a third case, an existing digital positive model may be available from when the reference prosthesis was made. In this case, neither block 1322 nor block 1324 are needed. The existing digital positive model may be used as input for block 1330 into the digital model for the new prosthetic limb that is to be created.

In some cases, method 1300 may include block 1326 of applying reference markers to the reference prosthetic limb, prior to the generating the scan of the reference prosthetic limb. That is, reference markers (i.e., reference marks, which may have datum points or datum marks on them) are placed on the reference prosthesis device. The reference markers can be useful because it is difficult to accurately align the reference socket (i.e., scan of the reference socket positive) to the scan of the reference prosthesis. The reference markers may also be used to assist in performing quality control of the reference prosthesis scan and can help the scanner monitor a relative position during the scan. Additionally, the scanning of the reference prosthesis in block 1320 may involve taking measurements, which may be done manually and/or digitally. Reference markers can provide datum marks for these measurements.

In some aspects, the reference markers may be markings made on the prosthetic device, such as with a pen or other writing instrument. In some aspects, the reference markers may be items or objects coupled to (e.g., adhered or attached with other techniques) to the reference prosthesis device. For example, the reference markers can be stickers (e.g., scanning dots/circles, “X” marks, or colored tape). In another example, the reference markers may be raised stickers, pins, spheres or other objects of other geometries that are, for example, adhered, pinned or clipped onto the prosthesis. In some aspects, the method may involve removing material, such as drilling the prosthesis to create holes to serve as reference marks. In some aspects, more than one type of reference marker may be utilized such as spheres on the brim of the reference socket and stickers on the pylon and/or foot.

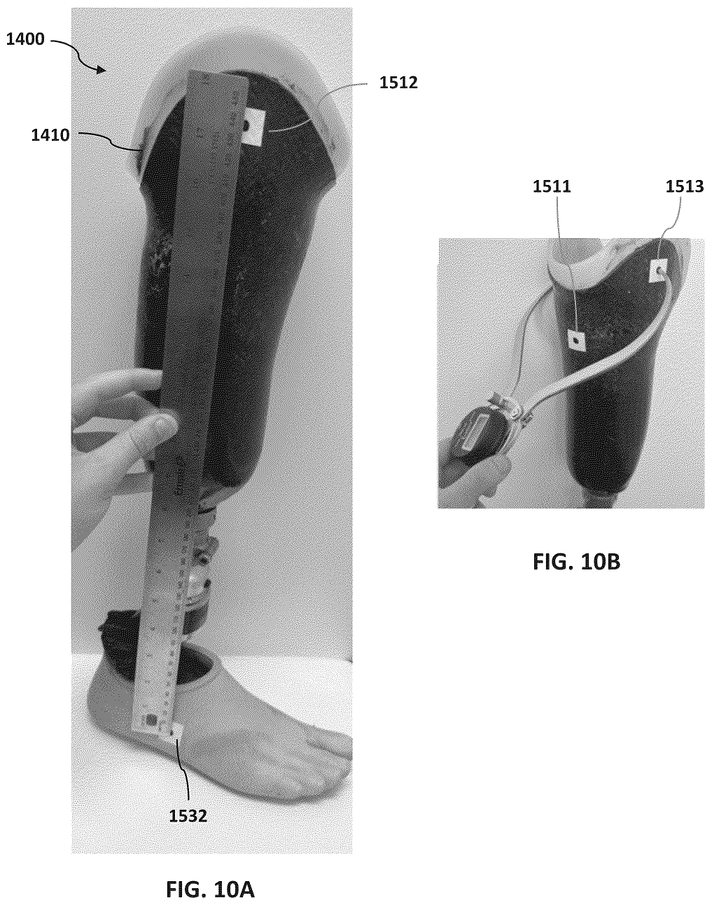

Various examples of reference markers are shown in FIGS. 9A-11D. In FIGS. 9A-10B, the reference markers are stickers or pieces of tape with a dot drawn or printed on the reference markers. FIGS. 9A and 10B show a reference marker 1511 applied on an anterior location on the socket 1410. FIGS. 9A and 9C show a reference marker 1512 mounted on a lateral location on the socket 1410 (e.g., on an car of the socket 1410 in this example). FIG. 9B shows a reference marker 1531 located on a medial side of the foot 1434. FIG. 9C shows a reference marker 1532 located on a lateral side of the foot 1434. FIG. 10B shows a reference marker 1513 coupled to a medial side of the socket 1410 (e.g., on an car of the socket 1410 in this example).

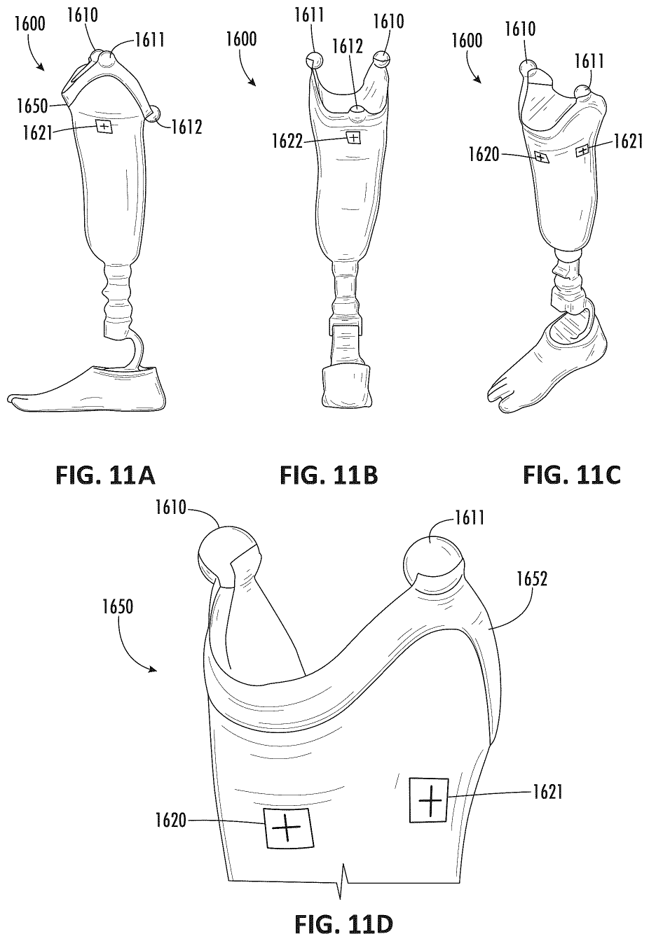

FIGS. 11A-11D show examples of reference markers that are objects attached (e.g., adhered or fastened) to a reference prosthetic limb, where the objects are spheres in these examples. FIGS. 11A-11C are medial, posterior, and perspective views, respectively, of an example reference prosthetic limb 1600, and FIG. 11D is a close-up view of the upper portion of a reference socket 1650 that is part of the reference prosthetic limb 1600. In some cases, reference socket 1650 may include an alginate or plaster socket positive 1652 that is cast (i.e., poured, molded) into the socket 1650 of reference prosthetic limb 1600. FIGS. 11A, 11B, and 11C show spherical reference markers 1610, 1611, and 1612 on lateral, medial, and posterior upper edges (i.e., brim) of the reference socket 1650 or the socket positive 1652. FIGS. 11A, 11B, and 11C also illustrate that multiple types of reference markers can be used simultaneously. Reference markers 1620, 1621 and 1622 on anterior, medial, and posterior locations, respectively, on an external surface of the reference prosthetic limb 1600 are stickers or tape attached to the socket area and may be color-based (e.g., a contrasting color from the socket/pylon). In this example, reference markers 1620, 1621 and 1622 have an “X” or “+” mark to serve as a datum mark or datum point.

The reference markers can be used in various ways. In one example, markers may be placed on anterior, posterior, medial, and/or lateral locations on the reference socket, such as on an exterior surface of the reference socket (reference markers 1620, 1621 and 1622) or on a brim of the reference socket (reference markers 1610, 1611, and 1612). Such reference markers on the socket can be used, for example, for determining AP and ML measurements of the socket and a height of the prosthetic limb. In another example, markers may be placed on a medial side (i.e., inner face) and/or a lateral side (outer face) of the prosthetic foot (e.g., FIGS. 9A, 9C), to help determine a width of the foot and/or height of the prosthetic limb. In a further example, physical markers can be used to maintain the scanner position (e.g., of the scanner application on a smartphone or computer tablet) to keep the scanner from losing track of position.

In some cases, if the socket of the reference prosthesis is copied by pouring a material such as plaster or alginate into the reference socket to make a physical positive model of the socket, markers can be placed on the poured positive while the physical positive model is still in the reference prosthesis (e.g., at the top of the poured socket per reference markers 1610, 1611 and 1612). Then scans may be taken of the entire prosthesis (external surface) including markers, and the socket positive removed and scanned to obtain a scan of the internal surface of the socket. In such cases, block 1322 involves pouring a material into the reference socket to make a physical positive model, and block 1326 involves placing a reference marker (e.g., one or more reference markers) on the material while the physical positive model is in the reference socket. Block 1324 involves scanning the reference socket while the physical positive model in the reference socket and scanning the physical positive model after removing the physical positive model from the reference socket. The markers in the scan of only the physical positive can be matched to the markers in the scan of the physical positive with the reference socket, thus aligning the physical positive scan within the prosthesis scan. Thus, the reference markers enable the alignment of the scan of the reference socket positive with the prosthesis scan in block 1330 of generating a digital model.

Another object that can be used as a reference marker is a pipe 1765 represented in the digital scans FIGS. 17A-17C. The pipe may be inserted while pouring an alginate socket, where the pipe extends a distance (e.g., 6 inches to 8 inches) above the top surface of the socket. The pipe can be used to align and/or make measurements when creating a digital model of the new prosthetic limb.

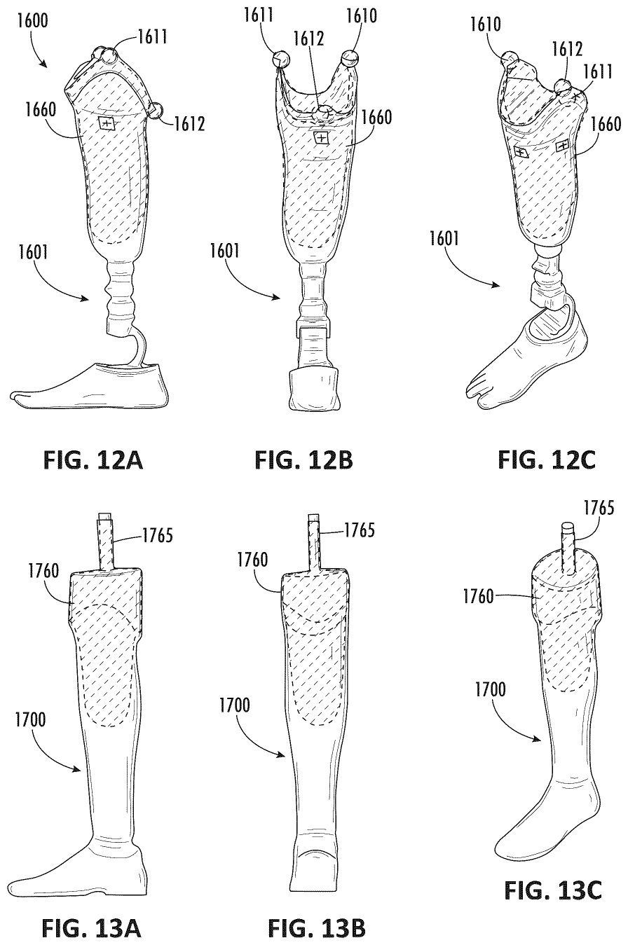

FIGS. 12A-12C show how a scan 1660 of a reference socket (e.g., reference socket positive 1652 of FIG. 11D) is aligned with a scan 1601 of the reference prosthetic limb during block 1330 of generating a digital model of a new transtibial prosthetic device. FIG. 12A is a medial view, FIG. 12B is a posterior view, and FIG. 12C is a perspective view. In this example, the scanned three physical spheres (reference markers 1610, 1611, and 1612) on the brim of the scan 1660 of the reference socket positive are superimposed on the identical places of the scanned reference prosthesis. The reference marker (sphere) locations are then used to align the scan 1660 of the reference socket to scan 1601 of the reference prosthetic limb.

FIGS. 13A-13C illustrate an example where scans of a reference socket and a reference prosthetic limb may be aligned without use of any reference markers. FIG. 13A is a medial view, FIG. 13B is a posterior view, and FIG. 13C is a perspective view. In these cases, the method may rely on matching or superimposing a reference socket scan 1760 with a scan 1700 of a reference prosthesis. The reference socket scan 1760 may be generated by scanning a pre-existing socket positive of the patient's reference prosthetic limb or may be a pre-existing digital model of the reference socket that was previously saved. FIGS. 13A-13C show that no reference markers are used in this example. Instead, a feature such as a pipe 1765 and/or other features such as a trim line, patellar loading bar, socket shape, or other anatomical features may be used to translate, align and rotate the socket positive (reference socket scan 1760) until it fits correctly within the scan 1700 of the reference prosthesis.

Returning to FIGS. 10A-10B, block 1320 of generating a scan of the reference prosthetic limb may involve taking manual measurements. In an example where a positive model of the socket is already available (i.e., a socket was not poured), two datum marks (location marks on reference markers, such as the dots on reference markers 1511 and 1512 or the X marks on reference markers 1620, 1621, 1622) may be placed on the socket ear centers (medial-lateral knee center “MLKC”), two datum marks may be placed anterior-posterior (AP, i.e., front and back) of the socket at the patellar-tendon-bearing (PTB), and two datum marks may be placed on the medial-lateral sides of the foot shell. FIGS. 9A-10B illustrate these types of datum mark (and reference marker) positionings. In another example where the socket is poured, two datum marks may be placed above the trimline on the medial-lateral (ML) aspects of the alginate, two datum marks may be placed above the trimline on the AP aspects of the alginate, and two dots may be placed on the ML sides of the foot shell.

Wherever the reference markers are placed, measurements may be taken based on the datum marks on the reference markers using manual tools such as a ruler (FIG. 10A), calipers (FIG. 10B), and/or other measuring tools such as optical scanners (e.g., laser tape measure, laser distance meter). A medial-lateral width of the exterior surface of the socket may be taken as shown in FIG. 10B using the center-to-center distance for the two ML datum marks (dots on reference markers 1512 and 1513). An anterior-posterior width of the exterior surface of the socket may be taken using the center-to-center distance for the two AP datum marks (dot on reference marker 1511 and a posterior marker, not shown). A height of the medial surface of the reference prosthetic limb may be taken using the distance between the medial foot shell datum mark to the medial socket/alginate datum mark. A height of the lateral surface of the reference prosthetic limb (FIG. 10A) may be taken using the distance between the lateral foot shell datum mark (dot on reference marker 1532) to the lateral socket/alginate datum mark (dot on reference marker 1512).

Block 1320 of method 1300 may optionally involve taking dimensional measurements of the reference prosthetic limb, wherein the dimensional measurements comprise one or more of a width of the reference foot, an anterior-posterior width of the reference socket, a medial foot-socket height of the reference prosthetic limb, and a lateral foot-socket height of the reference prosthetic limb. The measured and scanned data may be entered via, for example, a website application. Known techniques may be used to scale the 3D model representation of the reference prosthetic limb using the measured dimensions. The dimensional measurements may be used, for example, for quality control of the scan data. If the measurements do not match the scan data, a new set of scans can be taken, or the scan data can be scaled to match the measurements.

Referring again to FIG. 8, a 3D scan of the reference prosthetic limb in block 1320 is performed while the prosthesis is in the vertical position. Block 1320 includes a scan of the external surfaces of the reference prosthesis as well as the internal surface of the socket. The scan of block 1320 can be performed with a customized software application and/or 3D scanner (e.g., Comb application and Apple iphone XR True Depth Camera; or LIMBER™ Prosthetics & Orthotics software application), taking circumferential views to achieve a 3D scan.

For scanning the internal surface of the socket in block 1320, if a physical positive model of the reference socket is available, the physical model is scanned as was described above for block 1324. If the socket was poured to create a positive of the reference socket, the prosthesis is removed while leaving the positive socket in the milmo. The alginate positive is then scanned. If a digital model of the reference socket is already available, block 1324 may be omitted and the socket scan may be uploaded directly in block 1330.

Block 1330 involves generating a digital model for the transtibial prosthetic device, wherein the transtibial prosthetic device comprises i) a socket, ii) a pylon comprising a unitary truss structure, and iii) a foot-ankle complex, and wherein an alignment of the pylon with the socket and with the foot-ankle complex is based on the scan. The scan of the reference socket from block 1324 and the scan of the reference (patient's existing) pylon and reference foot-ankle prosthesis from block 1320 are brought together into a design software (e.g., mesh modeling software or other computer aided modeling software). Block 1330 may involve orienting the reference prosthesis scan in a digital design workspace and aligning the digital reference socket positive to the reference prosthesis scan. For example, the existing prosthesis may be moved into a vertical position and rotated so the medial border of the foot shell follows the line of progression. The reference socket is then positioned to match the existing prosthesis. To create a new transtibial prosthetic limb, the ankle-foot is positioned to match the existing (reference) foot shell. The pylon of the new transtibial prosthetic limb is positioned to properly join the top of the ankle with the bottom of the socket.

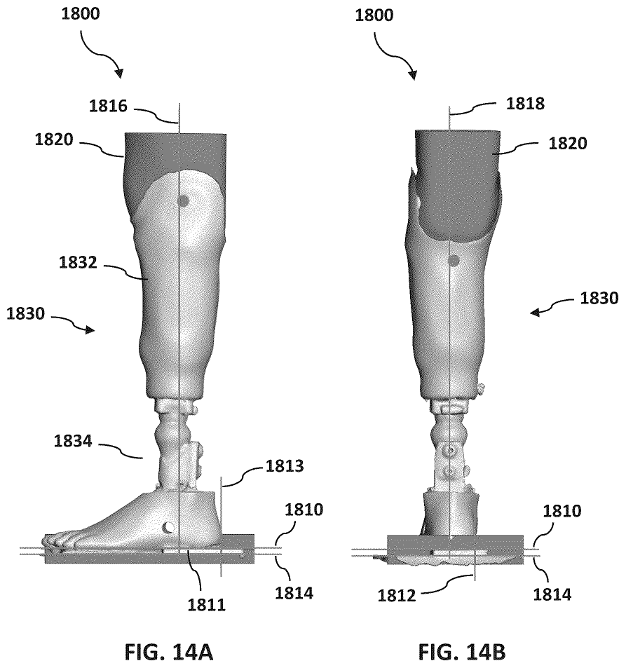

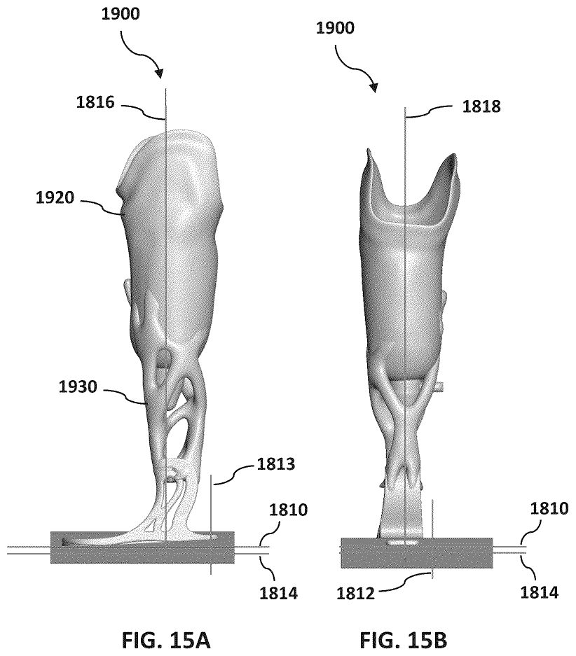

FIGS. 14A-14B and 15A-15B demonstrate “digital assembly” steps involved with generating a digital model of the transtibial prosthetic limb in block 1330. FIGS. 14A-14B are lateral and posterior views, respectively showing how a reference prosthesis scan 1800 is aligned with a set of planes. There is a heel wedge plane 1810, medial border plane 1812 (to set the medial border of the footshell), heel stop plane 1813 (to set the posterior position of the heel of the foot shell), and floor plane 1814. A heel wedge 1811 is used in this example, to help properly align the prosthesis during scanning. The scan of the socket positive 1820 is then aligned with the scan of the reference prosthesis 1830 which includes the reference socket 1832 and reference foot-ankle 1834. The coronal plane 1816 and sagittal plane 1818 are created based on the reference prosthesis. The coronal plane 1816 is based on the center of weight of the socket, while the sagittal plane 1818 is based on the center of weight of the foot.

FIGS. 15A-15B show how the pylon-ankle-foot of the transtibial prosthetic device 1900 (UniLeg), which includes a unitary truss structure in the pylon 1930, is digitally aligned with the various planes in block 1330 of the method 1300 (e.g., the same planes described for FIGS. 14A-14B). Relationships between the patient weight, patient foot length, patient height, patient activity level, and other parameters are utilized to determine how to place the foot with respect to the coronal plane 1816 and sagittal plane 1818. The socket 1920 is built directly off the reference socket positive scan.

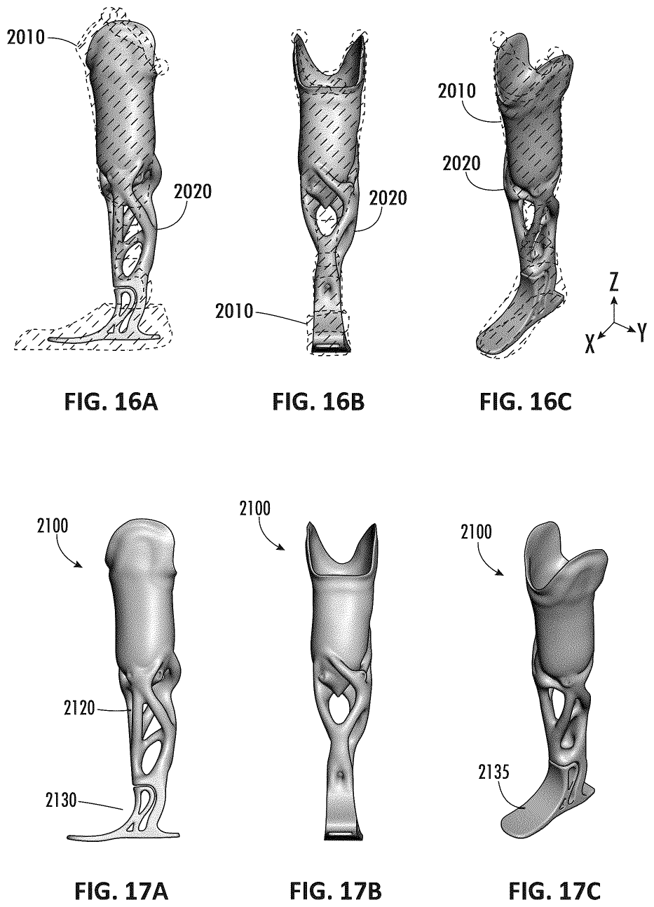

FIGS. 16A, 16B and 16C are lateral, posterior, and perspective views, respectively, that further illustrate generating the digital model of block 1330. In these figures, the scan 2010 of the reference prosthesis generated from block 1320 and the digital design 2020 of the new transtibial prosthetic device are overlaid on each other. The digital design 2020 is adjusted such that the alignment of the socket, pylon and foot-ankle of the reference prosthetic limb is transferred to the unibody transtibial prosthetic device (which includes a unitary pylon-ankle-foot and optionally a socket). FIGS. 16A-16C demonstrate the achievement of identical alignment between the reference prosthesis and new transtibial prosthetic device.

FIGS. 17A, 17B and 17C are lateral, posterior, and perspective views, respectively, of a resulting transtibial prosthetic device 2100 fabricated using the digital model and printing, where the pylon 2120 and the foot-ankle complex 2130 are a unitary single piece. The pylon 2120 is a unitary truss structure comprising interconnected elongated supports as described throughout this disclosure. The foot-ankle complex 2130 may have the s-shaped anterior and s-shaped posterior design for multi-axial dynamic flex as described throughout the disclosure. The transtibial prosthetic device 2100 has dimensions that have been scaled based on dimensions of the reference prosthetic limb, and on patient factors as described above (e.g., using patient data that was collected in block 1310). The assembled and integrated transtibial prosthetic device 2100 (UniLeg) of FIGS. 17A-17C is properly shaped and aligned based on the reference prosthesis.

In examples of block 1330 of method 1300, alignment of the pylon with the socket and the foot-ankle complex in the digital model includes superimposing the socket with the scan of the reference socket; positioning the foot-ankle complex based on the scan of the reference foot; and aligning a distal end of the pylon with a top of the foot-ankle complex and a proximal end of the pylon with a bottom of the socket. The unitary truss structure (e.g., transtibial prosthetic device 2100 in FIGS. 17A-17C) may be formed of a plurality of elongated supports interconnected at nodes as described throughout this disclosure (e.g., FIGS. 1A-1B, 3A-3C, 5A-5B). The foot-ankle complex (as described in, e.g., FIGS. 1A-1B, 4A-4C, 6) may include a sole portion; an s-shaped posterior portion extending from a first location on the sole portion to a base of the pylon; and an s-shaped anterior portion extending from a second location on the sole portion to a terminal portion of the s-shaped anterior portion near the base of the pylon, the terminal portion being unconnected to the base of the pylon.

In some cases, block 1330 involves scaling the height and width of the pylon in X, Y and Z directions (FIG. 16C) based on various parameters such as patient weight and activity level. In some cases, the size of the foot may be scaled based on patient foot size, and the blade 2135 (FIG. 17C) of the foot may be scaled based on patient weight and activity level. In some cases, the lengths and diameters of the elongated supports in the truss structure may be scaled according to patient parameters such as height, weight, and activity level and to accommodate the alignment transferred from the reference prosthesis. In some cases, the angles of the elongated supports in the trusses may also be adjusted. Also, the alignment, flex, and adduction of the transtibial prosthetic device may be based on how the patient presents, where the prosthetic device is customized to achieve the desired alignment.

Various parameters for customizing and adjusting the pylon-ankle-foot in the digital design of the transtibial prosthetic limb may include a desired stored energy, desired heel compression, desired heel height, patient weight, patient foot length, patient leg length, patient height, and/or activity K-level. In some cases, the design of the prosthetic leg can be customized for specific activities to be performed by the patient, such as weightlifting, hiking with a backpack, running, surfing, surfcasting, or fly-fishing. Other parameters for adjusting the pylon-ankle-foot may include the residual limb length and shape (which are also considerations for the socket, pylon, and alignment), and the residual limb alignment (e.g., overly flexed or overly adducted). As an example, heavier patient weights may require thicker or larger diameter supports in the unitary truss. As another example, certain types of activities (e.g., running, surfing) may require adjustments in strength and/or stiffness characteristics in particular parts of the foot and/or ankle

The foot of the transtibial prosthetic limb may be adjusted parametrically. For example, the thickness of certain parts such as the foot blade may be based on relationships with other parameters or dimensions. The relationships may be linear or non-linear. For instance, the thickness of the foot blade may scale non-linearly with foot length or weight of the patient.

Materials of different stiffnesses as described throughout this disclosure (e.g., in relation to FIGS. 6-7) may be incorporated into the transtibial prosthetic limb design to adjust energy return, heel compression, and flexibility based on factors such as the input weight, activity level, and specific activity requirements of the patient. For example, carbon fiber or other materials may be implemented to provide higher functionality for more active patients (e.g., based on the patient's specifics activity level/K-level).

Referring again to FIG. 8, block 1340 involves fabricating, using the digital model from block 1330 and 3D printing, the pylon and the foot-ankle complex as a unitary single piece. The printing may be performed as described for blocks 154 and 156 of FIG. 1F, where a print model (e.g., G-code) is generated and the prosthetic limb (e.g., of FIGS. 17A-17C) is printed. In some cases, the socket may be created using prosthetic design software Neo (Rodin4D, Italy). The pylon with its present bioinspired truss structure may be created using topology optimization software nTopology (nTopology, New York, NY) controlled to implement a pylon having a unitary bioinspired truss structure of interconnected elongated supports. The multi-axial dynamic foot-ankle complex may be created in Fusion360 (Autodesk, San Rafael, CA) controlled to implement a foot-ankle complex of the present disclosure that provides multi-axial dynamic flex. Finally, alignment and blending of the socket, pylon, and foot-ankle complex into the present unitary single-piece transtibial prosthesis may be done in Meshmixer (Autodesk, San Rafael, CA). The finished model may be sliced into G-code that the 3D printer can interpret using Simplify3D (Simplify3D, Cincinnati, OH). The fused filament fabrication (FFF) manufacturing process uses engineering grade thermoplastics to produce a strong and durable endoskeletal prosthetic device.

Between blocks 1330 and 1340, the design may be inspected. This inspection is done in mesh modeling software where the reference model (original prosthesis) and the designed model of the transtibial prosthetic limb (UniLeg transtibial prosthetic limb) are superimposed on each other to check proper alignment and height of the UniLeg. Design inspection may be performed to validate scan quality. Because different scanners have varying quality, the method 1300 may include comparing virtual measurements to manual measurements (e.g., distances between reference markers) made on the prosthesis before scanning. This enables dimensions of the scan to be corrected if there is a discrepancy.

Block 1350 involves fabricating a socket for the transtibial prosthetic device using the reference positive. In some cases, the socket is fabricated as a separate piece and then attached to the pylon-foot-ankle. In some cases, the socket is printed as a unitary (integral) piece with the pylon-foot-ankle. Various parameters for the adjustment (design) of the socket may include options for suspension types (e.g., suction, pin-lock, sleeve). The methods may include the ability to isolate and locate strength needs in the socket design, where material may be added to allow for joints and lacer attachment, reinforcement at a proximal aspect of the socket to counter varus valgus instability, or to provide extra material grinding (e.g., anterior distal tibia, fibular head).

In some cases, block 1350 may involve creating a socket from the 3D scan of the reference socket (e.g., from block 1324 or a pre-existing digital model). In block 1350, the socket may be configured to accommodate (i.e., accept, or fit) a desired suspension component. Any type of suspension component (e.g., shuttle lock, expulsion valve, pin-lock or other as described elsewhere in this disclosure) may be incorporated during the digital design process. FIGS. 18A-18C depict a socket configured to accommodate a pin-lock as a suspension component, where FIG. 18A is a side view of a socket 2200, FIG. 18B is a perspective view of FIG. 18B, and FIG. 18C is a view taken at plane A-A of FIG. 18B. In this example, the distal end 2210 of the socket 2200 has an aperture 2220 that is adapted to receive a pin-lock component (not shown). In this manner, the pin-lock component may be inserted through the top of the receptacle (i.e., mating negative) of the distal end 2210 of socket, and the button of the pin-lock component is threaded in through aperture 2220.

For each type of suspension component such as a pin-lock, expulsion valve, vacuum system, strap system, cable system, or other, specific features may be built into the socket as needed so that the socket may be attached to the UniLeg. When the prosthetist customer receives the transtibial prosthetic device (UniLeg), they can then install the specific suspension component into the socket via threads, bolts, rivets, adhesives, or other mechanism. Another example of a suspension component is a suspension sleeve, in which no socket modification is necessary in the design of the socket. That is, a suspension sleeve does not require any features incorporated into the socket of the UniLeg.

In the design of the transtibial device (during block 1330) any or all the components can be changed compared to the reference prosthesis while maintaining proper alignment, height, and other parameters. For example, the patient may want a stiffer foot or different socket design than the reference device. In another example, the patient may need their socket may need to be larger than the reference prosthetic limb to accommodate weight gain. In another example, additional height may be added to the socket. In such examples, a new socket can be designed using method 1300 to properly align the components. Inputs that may be used to design the socket include, for instance, a scan of the residual limb or rectified positive mold; circumference measurements; lengths of the fibula, tibia and residual limb; ML knee center; AP patellar tendon; amputation type, side of amputation, tissue level, and functional level; percent of desired reduction.

In block 1360, the unitary pylon and foot-ankle fabricated in block 1340 is assembled with the socket fabricated in block 1350, and the transtibial prosthetic device is completed. Further steps may include inspecting the final device and performing any testing and adjusting as needed.

### Experiment Observations

The demonstrated imaging and 3D printing workflows can be used to provide prosthetic devices to virtually any person cared by a practitioner that has a smart phone or other mobile device for imaging, which can benefit rural communities, where high-tech, medical imaging devices such as CT scanners are not available or cost-prohibitive. The workflow provides custom-fitted prostheses that are comfortable and high-performance.

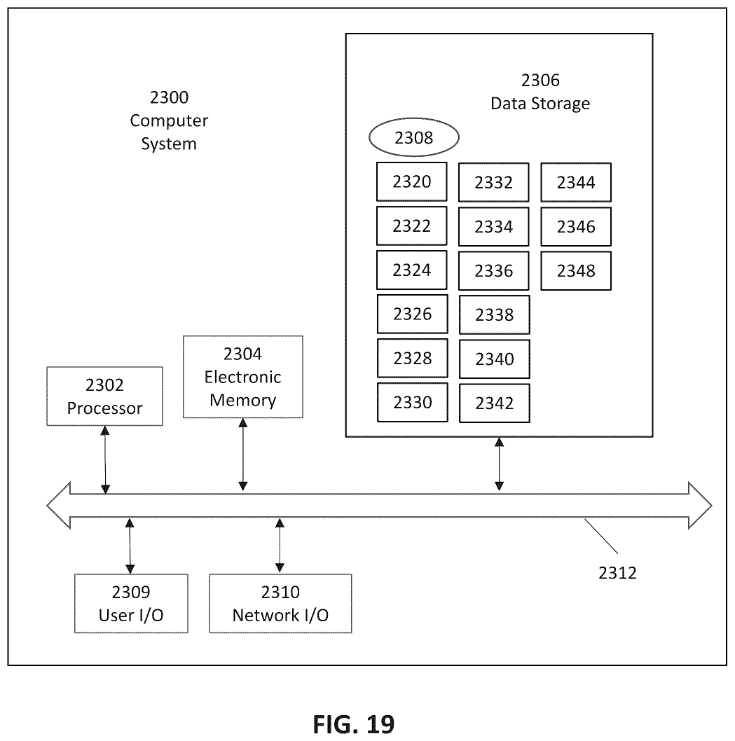

FIG. 19 is a simplified schematic diagram showing an example computer system 2300 (representing any combination of one or more of the computer systems) for use in the methods and systems of the present disclosure. The computer system 2300 represents one or more computer processors that may be used to perform various steps of the methods described herein, such as for generating scans of a reference socket or reference prosthetic limb, or generating a digital model of a transtibial prosthetic device. These various steps may be all be performed by one computer processor, or some steps may be performed by one computer processor while other steps are performed by another computer processor. The computer processor may be part of, for example, a smartphone, a computer tablet, or a computer workstation (e.g., desktop computer or laptop/notebook computer) that is used by a prosthetist or other professional designing the transtibial prosthetic device.

In the illustrated example, the computer system 2300 generally includes at least one processor 2302, at least one main electronic memory 2304, at least one data storage 2306, at least one user I/O 2309, and at least one network I/O 2310, among other components not shown for simplicity, connected or coupled together by a data communication subsystem 2312.

The processor 2302 represents one or more central processing units on one or more PCBs (printed circuit boards) in one or more housings or enclosures. In some examples, the processor 2302 represents multiple microprocessor units in multiple computer devices at multiple physical locations interconnected by one or more data channels. When executing computer-executable instructions for performing the above-described functions of the computer system 2300 in cooperation with the main electronic memory 2304, the processor 2302 becomes a special purpose computer for performing the functions of the instructions.

The main electronic memory 2304 represents one or more RAM modules on one or more PCBs in one or more housings or enclosures. In some cases, the main electronic memory 2304 represents multiple memory module units in multiple computer devices at multiple physical locations. In operation with the processor 2302, the main electronic memory 2304 stores the computer-executable instructions executed by, and data processed or generated by, the processor 2302 to perform the above-described functions of the computer system 2300.

The data storage 2306 represents or comprises any appropriate number or combination of internal or external physical mass storage devices, such as hard drives, optical drives, network-attached storage (NAS) devices, flash drives, etc. In some cases, the data storage 2306 represents multiple mass storage devices in multiple computer devices at multiple physical locations. The data storage 2306 generally provides persistent storage (e.g., in a non-transitory computer-readable or machine-readable medium 2308) for the programs (e.g., computer-executable instructions) and data used in operation of the processor 2302 and the main electronic memory 2304. The non-transitory computer readable medium 2308 includes instructions (e.g., the programs and data 2320, 2322, 2324, 2326, 2328, 2330, 2332, 2334, 2336, 2338, 2340, 2342, 2234, 2346, 2348) that, when executed by the processor 2302, cause the processor 2302 to perform operations including the above-described functions of the computer system 2300.

In some examples, the main electronic memory 2304 and the data storage 2306 include all, or a portion of the programs and data (e.g., represented by 2320-2348) required by the processor 2302 to perform the methods, processes and functions disclosed herein (e.g., in FIGS. 4-6). Under control of these programs and using this data, the processor 2302, in cooperation with the main electronic memory 2304, performs the above-described functions for the computer system 2300.

The user I/O 2309 represents one or more appropriate user interface devices, such as keyboards, pointing devices, displays, etc. In some examples, the user I/O 2309 represents multiple user interface devices for multiple computer devices at multiple physical locations. A system administrator, for example, may use these devices to access, set up, and control the computer system 2300.

The network I/O 2310 represents any appropriate networking devices, such as network adapters, etc., for communicating throughout the system. In some examples, the network I/O 2310 represents multiple such networking devices for multiple computer devices at multiple physical locations for communicating through multiple data channels.

The data communication subsystem 2312 represents any appropriate communication hardware for connecting the other components in a single unit or in a distributed manner on one or more PCBs, within one or more housings or enclosures, within one or more rack assemblies, within one or more geographical locations, etc.

Reference has been made in detail to aspects of the disclosed invention, one or more examples of which have been illustrated in the accompanying figures. Each example has been provided by way of explanation of the present technology, not as a limitation of the present technology. In fact, while the specification has been described in detail with respect to specific aspects of the invention, it will be appreciated that those skilled in the art, upon attaining an understanding of the foregoing, may readily conceive of alterations to, variations of, and equivalents to these aspects. For instance, features illustrated or described as part of one aspect may be used with another aspect to yield a still further aspect. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

1. A method for producing a transtibial prosthetic device, the method comprising:
generating a scan of a reference prosthetic limb, wherein the reference prosthetic limb has a reference socket, a reference pylon, and a reference foot;
generating a digital model for the transtibial prosthetic device, wherein the transtibial prosthetic device comprises i) a socket, ii) a pylon comprising a unitary truss structure, and iii) a foot-ankle complex, and wherein an alignment of the pylon with the socket and with the foot-ankle complex in the digital model is based on the scan; and
fabricating, using the digital model and 3D printing, the pylon and the foot-ankle complex as a unitary single piece.

2. The method of claim 1, wherein the reference socket, the reference pylon, and the reference foot have been assembled and aligned by a certified prosthetist/orthotist (CPO).

3. The method of claim 1, further comprising creating a physical positive model of the reference socket;
wherein the generating the scan of the reference prosthetic limb comprises scanning the physical positive model.

4. The method of claim 1, further comprising applying reference markers to the reference prosthetic limb, prior to the generating the scan of the reference prosthetic limb.

5. The method of claim 4, wherein the reference markers are placed on anterior, posterior, medial and lateral locations on the reference socket.

6. The method of claim 4, wherein the reference markers are placed on a medial side and a lateral side of the reference foot.

7. The method of claim 1, further comprising:
pouring a material into the reference socket to make a physical positive model;
placing a reference marker on the material while the physical positive model is in the reference socket;
scanning the reference socket while the physical positive model is in the reference socket; and
scanning the physical positive model after removing the physical positive model from the reference socket.

8. The method of claim 1, wherein the alignment of the pylon with the socket and the foot-ankle complex in the digital model comprises:
superimposing the socket with the scan of the reference socket;
positioning the foot-ankle complex based on the scan of the reference foot; and
aligning a distal end of the pylon with a top of the foot-ankle complex and a proximal end of the pylon with a bottom of the socket.

9. The method of claim 1, wherein the unitary truss structure is formed of a plurality of elongated supports interconnected at nodes.

10. The method of claim 1, wherein the foot-ankle complex comprises:
a sole portion;
an s-shaped posterior portion extending from a first location on the sole portion to a base of the pylon; and
an s-shaped anterior portion extending from a second location on the sole portion to a terminal portion of the s-shaped anterior portion near the base of the pylon, the terminal portion being unconnected to the base of the pylon.

11. The method of claim 1, further comprising taking dimensional measurements of the reference prosthetic limb, wherein the dimensional measurements comprise one or more of a width of the reference foot, an anterior-posterior width of the reference socket, a medial foot-socket height of the reference prosthetic limb, and a lateral foot-socket height of the reference prosthetic limb.

12. A method for producing a unibody transtibial prosthetic device, the method comprising:
acquiring patient data via imaging and/or scanning;
constructing a 3D model from the patient data;
translating the 3D model to 3D printable design of a unibody transtibial prosthesis; and
3D printing the unibody transtibial prosthesis;
wherein:
the unibody transtibial prosthesis comprises a pylon and a foot-ankle complex;
the pylon has a unitary truss structure formed of a plurality of elongated supports interconnected at nodes; and
the foot-ankle complex extends from a base of the pylon and comprises: i) a sole portion; ii) an s-shaped posterior portion extending from a first location on the sole portion to the base of the pylon; and iii) an s-shaped anterior portion extending from a second location on the sole portion to a terminal portion of the s-shaped anterior portion near the base of the pylon, the terminal portion being unconnected to the base of the pylon.

13. The method of claim 12, wherein the acquiring patient data comprises imaging a contralateral limb and residual limb.

14. The method of claim 12, wherein the constructing the 3D model comprises shaping and dimensioning the unibody transtibial prosthesis with topology optimization to be lightweight in a total amount of polymer material for a given targeted K-level of use.

15. The method of claim 12, wherein the unibody transtibial prosthesis further comprises a socket; and
wherein the constructing the 3D model comprises aligning and blending the socket, the pylon, and the foot-ankle complex.

16. The method of claim 12, wherein the unitary truss structure is an endoskeletal design; and
the method further comprises thermoforming to adjust alignment of the pylon.
